SEQUENCE LISTING (10 sequences)

SEQ ID NO: 1 - DNA, 2451 bp, Zea mays
  CDS (226)...(2175)
  misc_feature (2404)...(2404): n = a, t, c, or g
acccacgcgtccgcggagaaggcgtcctcgacgactgcccatggccgccgcccggacctaggctagggattgctgtctcctgagtcccgacctaccaaggcggaaagggagagggggcgctgtggcgtcaggaaataactggaagccgagggctgagggcgagttcagagtcaggtggttatcgctttctttttcgttttctttttccggagggtagtattggggatgccggttgctaggccggagccgcaggagccccgggtgatcgcccatgtcgacatggactgcttctatgttcaagtcgagcagcggaggaacccggcgctcaggggacagccgaccgccgtggtgcagtacaacggctggaaaggcggcggcttgattgccgtcagctacgaggctcgtggatttggtgtgaagaggtccatgcgcggagatgaggccaagagggtttgtcctggtattaatcttgttcaggtcccagtggcgcgtggcaaggctgaccttaatctttacagaagtgctggtgctgaggttgttgcaatccttgcaagcaaagggaagtgcgagcgggcatccatcgacgaagtttatcttgaccttactgatgcagcaaaggaaatgctcttacaagctcccccagattcaccagaggggatttttatggaggcggcaaagtcaaatatcttgggccttccggctgatgccagcgagaaggaaaagaatgtgagggcatggctttgtcaatcagaagctgattaccaggacaagttactggcatgtggagctataattgttgcacagttacgagtcagagttctggaggaaacccaattcacatgctctgctgggattgctcacaataagatgttagctaaacttgtcagtggaatgcacaagcctgctcagcaaacagttgtcccttcttcatcagttcaagacttactagcatcactacctgtgaagaagatgaaacagcttggtggtaagcttggaagttccttgcaggatgatcttggggttgagaccattggtgatctcctaagttttacagaggaaaaattacaagagcagtatggagtaaatactggaacttggttatggaagactgccaggggcattagcggggaagaagttgaggaccgtcttctcccaaagagtcacgggtgtggaaagacatttcctggcccaagagcactgaagtacagtgcttctgtcaaaggctggctggatcaactatgtgaagaattaagtgaacgtatccagtctgacctgaaccagaacaagagaattgctcaaacgctaactcttcatgccagagcatttaagaaaaatgaacatgattcaatgaagaaattcccttccaaatcctgtcctttgcgctatgggactgggaaaattcaggaggatgcaatgaggctatttgaatctggccttcatgagtttttagaatctcagaacactggatggggcataacatctctttctgtcactgcaagcaaaatatttgatatacctagtggaacaagctcaatcctgagatacattaaaggtccaagttctgctgcagctctgactatacctgattctccaagttctgctgcagctctggctatacctgattcttcatttgtacctgaggatccatctcttgataatgatgtatttgtggagccaattcatgaagaagaatgccaaccatcaacctctgagaaagaagacgacaataatacacattctgcgtctgcattttcagctaaaaaatgccgagcaaatgaagaaaaaagaatatcaaagaaattgcctggagttcagggaacttcctcgatattgaagtttctttcacggggccaatctaccttacatgagaaaagaaaatctgatggactaatttgcagtcatcaaggtcccgggagttcttcggaagcatacaaagctggagcacacaatgtgcccgcggaagctgaggataggaacaacaccaacagttgtgctgaaccctctggcagtaacacatggacgttcaaccttcaagacattgatccagcggtggtagaggagctgccaccagagatccaaagagaaatacaggggtgggtccgtccatcgaagcacccgatcacgaagagacgtggttctacaatttcttcatactttccacccgcgaggtcttagatacgttgatacgttagttcttgattgtatattctttattttctacacacaacggcctcgctctgtgtattgtaattgtaagatgtgaagatgtttaggtagccctatatgtgattaacagcttgacgggacctctgagaatggtaagtttatttacgtggggtatggaaacatgaacattaaagcatgtgcaattgagttgaggtcctagggttgcatctaaaaaaancaaaaaaaaaaaaaaaaaaaaaaaaacaaaaaaaaaaaaaaaaaaaa

SEQ ID NO: 2 - protein, 649 aa, Zea mays
MPVARPEPQEPRVIAHVDMDCFYVQVEQRRNPALRGQPTAVVQYNGWKGGGLIAVSYEARGFGVKRSMRGDEAKRVCPGINLVQVPVARGKADLNLYRSAGAEVVAILASKGKCERASIDEVYLDLTDAAKEMLLQAPPDSPEGIFMEAAKSNILGLPADASEKEKNVRAWLCQSEADYQDKLLACGAIIVAQLRVRVLEETQFTCSAGIAHNKMLAKLVSGMHKPAQQTVVPSSSVQDLLASLPVKKMKQLGGKLGSSLQDDLGVETIGDLLSFTEEKLQEQYGVNTGTWLWKTARGISGEEVEDRLLPKSHGCGKTFPGPRALKYSASVKGWLDQLCEELSERIQSDLNQNKRIAQTLTLHARAFKKNEHDSMKKFPSKSCPLRYGTGKIQEDAMRLFESGLHEFLESQNTGWGITSLSVTASKIFDIPSGTSSILRYIKGPSSAAALTIPDSPSSAAALAIPDSSFVPEDPSLDNDVFVEPIHEEECQPSTSEKEDDNNTHSASAFSAKKCRANEEKRISKKLPGVQGTSSILKFLSRGQSTLHEKRKSDGLICSHQGPGSSSEAYKAGAHNVPAEAEDRNNTNSCAEPSGSNTWTFNLQDIDPAVVEELPPEIQREIQGWVRPSKHPITKRRGSTISSYFPPARS

SEQ ID NO: 3 - DNA, 36 bp, Artificial Sequence
  Synthetic SalA20 oligonucleotide
tcgacccacgcgtccgaaaaaaaaaaaaaaaaaaaa

SEQ ID NO: 4 - DNA, 20 bp, Artificial Sequence
  PCR primer GSTFOR
tccaaaagagcgtgcagaga

SEQ ID NO: 5 - DNA, 32 bp, Artificial Sequence
  PCR primer pGEXCFR
gctgggaccggcatgggcccctggaacagaac

SEQ ID NO: 6 - DNA, 23 bp, Artificial Sequence
  Primer pGEX5
gggctggcaagccacgtttggtg

SEQ ID NO: 7 - DNA, 33 bp, Artificial Sequence
  PCR primer PNFor
gtacgtgccatggggatgccggttgctaggccg

SEQ ID NO: 8 - DNA, 33 bp, Artificial Sequence
  PCR primer PNRev
cgccgatgcggccgcctaagacctcgcgggtgg

SEQ ID NO: 9 - DNA, 47 bp, Artificial Sequence
  Linker strand YLTOP
tcgaggcggtggcggccgctcgtggatcccgtcgaccaggaattcgt

SEQ ID NO: 10 - DNA, 47 bp, Artificial Sequence
  Primer YLBottom
ctagacgaattcctggtcgacgggatccacgagcggccgccaccgcc

(19) United States
(12) Patent Application Publication
(10) Pub. No.: US 2003/0084476 A1
(43) Pub. Date: May 1, 2003

(54) DNA polymerase eta ( Poleta) cDNA and uses thereof

(75) Inventors: Pramod B. Mahajan, Urbandale, IA (US); Priya Kannan, Ankeny, IA (US)

Correspondence Address: PIONEER HI-BRED INTERNATIONAL INC., 7100 N.W. 62ND AVENUE, P.O. BOX 1000, JOHNSTON, IA, 50131, US

(73) Assignee: Pioneer Hi-Bred International, Inc.

(21) Appl. No.: 10/282,602

(22) Filed: Oct. 29, 2002

Related U.S. Application Data
(60) Provisional application No. 60/350,988, filed on Oct. 29, 2001.

(51) Int. Cl. A01H 5/00; C12Q 1/68; C07H 21/04; C12N 9/22; C12N 15/82; C12P 21/02; C12N 5/04

(52) U.S. Cl. 800/278; 800/312; 800/320.1; 435/6; 435/199; 435/69.1; 435/419; 435/320.1; 536/23.2

(57) ABSTRACT

The invention provides isolated RAD30/Polη nucleic acids and their encoded proteins. The present invention provides methods and compositions relating to altering RAD30/Polη levels in plants. The invention provides methods and compositions relating to introducing specific, heritable modifications to a target polynucleotide of interest. The invention further provides recombinant expression cassettes, host cells, transgenic plants, and antibody compositions.

### CROSS-REFERENCE TO RELATED APPLICATIONS

This application claims the benefit of U.S. Application Serial No. 60/350,988 filed Oct. 29, 2001, which is herein incorporated by reference.

### TECHNICAL FIELD

The present invention relates generally to plant molecular biology. More specifically, it relates to nucleic acids and methods for modulating their expression in plants.

### BACKGROUND OF THE INVENTION

Various environmental agents such as y-radiation, UV light in the 320-380 nm range, ozone, heat, and different chemicals cause oxidative damage to cellular DNA. Similarly, reactive oxygen species, hydroxyl radicals and superoxide and nitric oxide species generated in vivo also cause oxidative damage to DNA (Friedberg, E. et al. (1995) in DNA Repair and Mutagenesis pp. 14-19 American Society of Microbiology Press, Washington D.C.). The precise DNA damage varies depending on the exposure and type of causative reagent. For example, γ-rays cause double-strand breaks and exposure to UV light results in formation of Tˆ T dimers. This damage can lead to genomic instability if not repaired. Consequently, all living organisms have developed specific enzymatic pathways to remove these lesions and maintain genomic stability.

In addition to specific enzymatic activities responsible for removal and repair of the DNA damage, biochemical and genetic investigations in prokaryotes uncovered an interesting mechanism of overcoming DNA damage (reviewed by Hatahet, Z. and Wallace, S. 1998, in DNA Damage and Repair: DNA Repair in Prokaryotes and Lower Eukaryotes Vol 1, pp. 229-262, Eds. Nickoloff and Hoekstra, Humana Press, Totowa, N.J.). This pathway involves de novo synthesis of DNA using the damaged DNA as the template to accurately synthesize DNA of the correct sequence. This damage bypass repair is called translesion synthesis (abbreviated hereafter as TLS). Enzymes involved in this pathway belong to a very large gene family that spans prokaryotes and eukaryotes, the UmuC/DinB/RAD30/Polη gene family, wherein the UmuC and DinB genes were characterized from E. coli, RAD30 from S. cerevisiae and Polη from human and mouse. Members of this superfamily share important structural motifs that are critical for their TLS function, and are conserved from bacteria to humans (McDonald J P et al. (1997) Genetics, 147:1557-1568; Gerlach V L et al. (1999) PNAS (USA), 96:11922-11927). Many of these genes encode specific DNA polymerases (Johnson R E et al. (1999) PNAS (USA) 96:12224-12226) characterized by high fidelity of replication across a lesion, but poor fidelity and low processivity on undamaged DNA. The UmuC/DinB/RAD30/Polη gene family has been divided into four sub-families. One of these subfamilies, Rad30/Polη is represented by the S. cerevisiae RAD30 gene (McDonald J P et al. (1997) Genetics 147:1557-156; Johnson R E et al. (1999) Science 283:1001-1004; Johnson R E et al. (1999) J Biol Chem 274:15975-15977); human Polη gene (Masutani C et al. (1999) Nature 399:700-704; McDonald J P et al. (1999) Genomics 60:20-30); and mouse Polη gene (McDonald J P et al. (1999) supra) orthologues. There are no published reports of Rad30/Polη homologues from plants.

In view of the unique ability of the DNA polymerases encoded by the RAD30/Polη subfamily to support faithful translesion DNA synthesis, these could be very valuable tools for targeted gene modification experiments. Presently, the methods available for oligonucleotide mediated in vivo targeted modifications of plant genes (for example, chimeraplasty) suffer from low efficiency.

### SUMMARY OF THE INVENTION

Control of DNA repair by the modulation of RAD30/Polη provides a means to induce or suppress DNA repair, or to create targeted polynucleotide sequence modifications. The ability of RAD30/Polη to support translesion DNA synthesis can be used to create targeted modifications by constructing template oligonucleotides comprising specific modified DNA lesions which will direct targeted changes at specific residues in a nucleic acid sequence of interest. Control of these processes has important implications in the creation of novel recombinantly engineered crops such as maize. The present invention provides this and other advantages.

The present invention teaches plant orthologues of RAD30/Polη polynucleotides and proteins. The present invention also teaches methods for modulating, in a transgenic plant, expression of the nucleic acids of the present invention. The present invention further teaches methods for in situ targeted sequence modification of a target polynucleotide of interest. In other aspects the present invention relates to: 1) recombinant expression cassettes, comprising a nucleic acid of the present invention operably linked to a promoter, 2) a host cell into which has been introduced the recombinant expression cassette, and 3) a transgenic plant comprising the recombinant expression cassette. The present invention also provides transgenic seed from the transgenic plant.

### DETAILED DESCRIPTION OF THE INVENTION

Definitions

Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges recited within the specification are inclusive of the numbers defining the range and include each integer within the defined range. Amino acids may be referred to herein by either their commonly known three letter symbols or by the one-letter symbols recommended by the IUPAC-IUBMB Nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5^{th} edition, 1993). The terms defined below are more fully defined by reference to the specification as a whole. Section headings provided throughout the specification are not limitations to the various objects and embodiments of the present invention.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, (1993) D. H. Persing et al., Ed., American Society for Microbiology, Washington, D.C. The product of amplification is termed an amplicon.

The term “antibody” includes reference to antigen binding forms of antibodies (e.g., Fab, F(ab)₂). The term “antibody” frequently refers to a polypeptide substantially encoded by an immunoglobulin gene or immunoglobulin genes, or fragments thereof which specifically bind and recognize an analyte (antigen). However, while various antibody fragments can be defined in terms of the digestion of an intact antibody, one of skill will appreciate that such fragments may be synthesized de novo either chemically or by utilizing recombinant DNA methodology. Thus, the term antibody, as used herein, also includes antibody fragments such as single chain Fv, chimeric antibodies (i.e., comprising constant and variable regions from different species), humanized antibodies (i.e., comprising a complementarity determining region (CDR) from a non-human source) and heteroconjugate antibodies (e.g., bispecific antibodies).

The term “antigen” includes reference to a substance to which an antibody can be generated and/or to which the antibody is specifically immunoreactive. The specific immunoreactive sites within the antigen are known as epitopes or antigenic determinants. These epitopes can be a linear array of monomers in a polymeric composition—such as amino acids in a protein—or consist of or comprise a more complex secondary or tertiary structure. Those of skill will recognize that all immunogens (i.e., substances capable of eliciting an immune response) are antigens; however some antigens, such as haptens, are not immunogens but may be made immunogenic by coupling to a carrier molecule. An antibody immunologically reactive with a particular antigen can be generated in vivo or by recombinant methods such as selection of libraries of recombinant antibodies in phage or similar vectors. See, e.g., Huse et al. (1989) Science 246:1275-1281; Ward et al. (1989) Nature 341:544-546; and Vaughan et al. (1996) Nature Biotech. 14:309-314.

As used herein, “antisense orientation” includes reference to-a duplex polynucleotide sequence that is operably linked to a promoter in an orientation where the antisense strand is transcribed. The antisense strand is sufficiently complementary to an endogenous transcription product such that translation of the endogenous transcription product is often inhibited.

As used herein, “chromosomal region” includes reference to a length of a chromosome that may be measured by reference to the linear segment of DNA that it comprises. The chromosomal region can be defined by reference to two unique DNA sequences, i.e., markers.

The term “conservatively modified variants” applies to both amino acid and nucleic acid sequences. With respect to particular nucleic acid sequences, conservatively modified variants refers to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the present invention.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant”. An alteration which results in the substitution of an amino acid with a chemically similar amino acid is also a conservatively modified variant. Thus, any number of amino acid residues selected from the group of integers consisting of from 1 to 15 or more can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

See also, Creighton (1984) Proteins, W. H. Freeman and Company.

As used herein, a “nucleic acid modification template” or “modification template” is a polynucleotide which contains nucleotide changes at specific locations within its sequence when compared to the DNA sequence of a “target” polynucleotide of interest. The modification template can be used to incorporate these nucleotide changes into the nucleic acid sequence of the target sequence in order to effect a “targeted modification” event. The modification template is typically homologous to the target polynucleotide of interest, except at the locations comprising the nucleotide changes to be incorporated. This homology directs the modification template to the polynucleotide of interest. The modification template may be comprised of DNA alone, or may be a DNA:RNA chimera as well as a PNA or other modified nucleotide polymer. The targeted modification will produce a heritable change in the target polynucleotide of interest.

As used herein, a “Tˆ T dimer” is a cis-syn cyclobutane photodimer stereoisomer of two thymidine nucleotides. This is the only physiologically relevant stereoisomer of a thymidine dimer.

By “encoding” or “encoded”, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as are present in some plant, animal, and fungal mitochondria, the bacterium Mycoplasma capricolum, or the ciliate Macronucleus, may be used when the nucleic acid is expressed therein.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledons or dicotyledons as these preferences have been shown to differ (Murray et al. (1989) Nucl. Acids Res. 17:477-498). Thus, the maize preferred codon for a particular amino acid may be derived from known gene sequences from maize. Maize codon usage for 28 genes from maize plants is listed in Table 4 of Murray et al. (1989), supra.

As used herein “full-length sequence” in reference to a specified polynucleotide or its encoded protein means having or encoding the entire amino acid sequence of a native (non-synthetic), endogenous, biologically (e.g., structurally or catalytically) active form of the specified protein. Methods to determine whether a sequence is full-length are well known in the art including such exemplary techniques as northern or western blots, primer extension, S1 protection, and ribonuclease protection. See, e.g., Plant Molecular Biology: A Laboratory Manual, Clark, Ed., Springer-Verlag, Berlin (1997). Comparison to known full-length homologous (orthologous and/or paralogous) sequences can also be used to identify full-length sequences of the present invention. Additionally, consensus sequences typically present at the 5′ and 3′ untranslated regions of mRNA aid in the identification of a polynucleotide as full-length. For example, the consensus sequence ANNNNAUGG, where the underlined codon represents the N-terminal methionine, aids in determining whether the polynucleotide has a complete 5′ end. Consensus sequences at the 3′ end, such as polyadenylation sequences, aid in determining whether the polynucleotide has a complete 3′ end.

As used herein, “heterologous” in reference to a nucleic acid is a nucleic acid that originates from a foreign species, or, if from the same species, is substantially modified from its native form in composition and/or genomic locus by human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by human intervention.

By “host cell” is meant a cell which contains a vector and supports the replication and/or expression of the vector. Host cells may be prokaryotic cells such as E. coli, or eukaryotic cells such as yeast, insect, amphibian, or mammalian cells. Host cells can also be monocotyledonous or dicotyledonous plant cells, an example of a monocotyledonous host cell is a maize host cell.

The term “hybridization complex” includes reference to a duplex nucleic acid structure formed by two single-stranded nucleic acid sequences selectively hybridized with each other.

The term “introduced” includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA). The term includes such nucleic acid introduction means as “transfection”, “transformation” and “transduction”.

The term “isolated” refers to material, such as a nucleic acid or a protein, which is substantially free from components that normally accompany or interact with it as found in its naturally occurring environment. The isolated material optionally comprises material not found with the material in its natural environment, or if the material is in its natural environment, the material has been altered by human intervention to a composition and/or a location in the cell (e.g., genome or subcellular organelle) not native to a material found in that environment. The alteration can be performed on the material within or removed from its natural state. For example, a naturally occurring nucleic acid becomes an isolated nucleic acid if it is altered, or if it is transcribed from DNA which has been altered, by means of human intervention performed within the cell from which it originates. See, e.g., Compounds and Methods for Site Directed Mutagenesis in Eukaryotic Cells, Kmiec, U.S. Pat. No. 5,565,350; In Vivo Homologous Sequence Targeting in Eukaryotic Cells; Zarling et al., PCT/US93/03868. Likewise, a naturally occurring nucleic acid (e.g., a promoter) becomes isolated if it is introduced by non-naturally occurring means to a locus of the genome not native to that nucleic acid. Nucleic acids which are “isolated” as defined herein, are also referred to as “heterologous” nucleic acids.

As used herein, “localized within the chromosomal region defined by and including” with respect to particular markers includes reference to a contiguous length of a chromosome delimited by and including the stated markers.

As used herein, “marker” includes reference to a locus on a chromosome that serves to identify a unique position on the chromosome. A “polymorphic marker” includes reference to a marker which appears in multiple forms (alleles) such that different forms of the marker, when they are present in a homologous pair, allow transmission of each of the chromosomes of that pair to be followed. A genotype may be defined by use of one or a plurality of markers.

As used herein, “nucleic acid” is used interchangably with the term “polynucleotide” and includes reference to a deoxyribopolynucleotide, ribopolynucleotide, or chimeras or analogs thereof that have the essential nature of a natural deoxy- or ribo-nucleotide in that they hybridize, under stringent hybridization conditions, to substantially the same nucleotide sequence as naturally occurring nucleotides and/or allow translation into the same amino acid(s) as the naturally occurring nucleotides (e.g., peptide nucleic acids). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

By “nucleic acid library” is meant a collection of isolated DNA or RNA molecules which comprise and substantially represent the entire transcribed fraction of a genome of a specified organism, tissue, or of a cell type from that organism. Construction of exemplary nucleic acid libraries, such as genomic and cDNA libraries, is taught in standard molecular biology references such as Berger and Kimmel, Guide to Molecular Cloning Techniques, Methods in Enzymology, Vol. 152, Academic Press, Inc., San Diego, Calif. (Berger); Sambrook et al., (1989) Molecular Cloning—A Laboratory Manual, 2nd ed., Vol. 1-3; and Current Protocols in Molecular Biology, F. M. Ausubel et al., Eds., Current Protocols, a joint venture between Greene Publishing Associates, Inc. and John Wiley & Sons, Inc. (1994).

As used herein “operably linked” includes reference to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

As used herein, the term “plant” includes reference to whole plants, plant organs (e.g., leaves, stems, roots, etc.), seeds and plant cells and progeny of same. Plant cell, as used herein includes, without limitation, seeds, suspension cultures, embryos, meristematic regions, callus tissue, leaves, roots, shoots, gametophytes, sporophytes, pollen, and microspores. The class of plants which can be used in the methods of the invention include both monocotyledonous and dicotyledonous plants. An example of a monocotyledonous plant is Zea mays.

The terms “polypeptide”, “peptide” and “protein” are used interchangeably herein to refer to a polymer of amino acid residues. The terms also apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation. Further, this invention contemplates the use of both the methionine-containing and the methionine-less amino terminal variants of the protein of the invention.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells whether or not its origin is a plant cell.

As used herein “RAD30/Pol” refers to a subfamily of the UmuC/DinB/RAD30/Polη family of DNA damage bypass replicative enzymes capable of translesion synthesis. This term refers to polynucleotides and polypeptides in their full-length form, as well as variants and functional fragments. In reference to the compositions and methods of the present invention the terms “Rad30”, ‘Polη” and ‘Rad30/Polη” can be used interchangably.

As used herein a “RAD30/Polη polynucleotide” is a polynucleotide of the present invention that encodes a polypeptide with RAD30/Polη translesion synthesis activity or that modulates the expression of RAD30/Polη mRNA or protein in host cells. The term RAD30/Polη polynucleotide includes subsequences or modified sequences of the polynucleotide sequences of the present invention.

As used herein a “RAD30/Polη polypeptide” is a polypeptide which modulates de novo synthesis of DNA using the damaged DNA as a template to accurately synthesize the correct DNA sequence, also known as translesion synthesis. The term RAD30/Polη polypeptide also includes fragments or modified sequences which retain the specific functional activity. The level of functional activity may be more than or less than the activity detected in a cellular extract comprising the endogenous enzyme.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under-expressed or not expressed at all as a result of human intervention. The term “recombinant” as used herein does not encompass the alteration of the cell or vector by naturally occurring events (e.g., spontaneous mutation, natural transformation/transduction/transposition) such as those occurring without human intervention.

As used herein, a “recombinant expression cassette” is a nucleic acid construct, generated recombinantly or synthetically, with a series of specified nucleic acid elements which permit transcription of a particular nucleic acid in a host cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter. It is recognized that the nucleic acid to be transcribed can be operably linked to a promoter in either a sense or an antisense orientation.

The term “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass non-natural analogs of natural amino acids that can function in a similar manner as naturally occurring amino acids.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, or 90% sequence identity, up to 100% sequence identity (i.e., complementary) with each other.

The term “stringent conditions” or “stringent hybridization conditions” includes reference to conditions under which a probe will selectively hybridize to its target sequence, to a detectably greater degree than to other sequences (e.g., at least 2-fold over background). Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1× SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284:

Tₘ=81.5° C.+16.6 (log M)+0.41 (% GC)−0.61 (% form)−500/L

where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. Hybridization and/or wash conditions can be applied for at least 10, 30, 60, 90, 120, or 240 minutes. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York (1993); and Current Protocols in Molecular Biology, Chapter 2, Ausubel et al., Eds., Greene Publishing and Wiley-Interscience, New York (1995).

As used herein, “transgenic plant” includes reference to a plant which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

As used herein, “vector” includes reference to a nucleic acid used in introduction of a polynucleotide of the present invention into a host cell. Vectors are often replicons. Expression vectors permit transcription of a nucleic acid inserted therein.

The following terms are used to describe the sequence relationships between a polynucleotide/polypeptide of the present invention with a reference polynucleotide/polypeptide: (a) “reference sequence”, (b) “comparison window”, (c) “sequence identity”, and (d) “percentage of sequence identity”.

(a) As used herein, “reference sequence” is a defined sequence used as a basis for sequence comparison with a polynucleotide/polypeptide of the present invention. A reference sequence may be a subset or the entirety of a specified sequence; for example, as a segment of a full-length cDNA or gene sequence, or the complete cDNA or gene sequence.

(b) As used herein, “comparison window” includes reference to a contiguous and specified segment of a polynucleotide/polypeptide sequence, wherein the polynucleotide/polypeptide sequence may be compared to a reference sequence and wherein the portion of the polynucleotide/polypeptide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides/amino acids residues in length, and optionally can be 30, 40, 50, 100, or longer. Those of skill in the art understand that to avoid a high similarity to a reference sequence due to inclusion of gaps in the polynucleotide/polypeptide sequence, a gap penalty is typically introduced and is subtracted from the number of matches.

Methods of alignment of sequences for comparison are well-known in the art. Optimal alignment of sequences for comparison may be conducted by the local homology algorithm of Smith and Waterman (1981) Adv. Appl. Math. 2:482; by the homology alignment algorithm of Needleman and Wunsch (1970) J. Mol. Biol. 48:443; by the search for similarity method of Pearson and Lipman (1988) PNAS (USA) 85:2444; by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA. The CLUSTAL program is well described by Higgins and Sharp (1988) Gene 73:237-244; Higgins and Sharp (1989) CABIOS 5:151-153; Corpet et al. (1988) Nucl Acids Res 16:10881-90; Huang et al. (1992) Computer Applications in the Biosciences 8:155-65, and Pearson et al. (1994) Methods in Molecular Biology 24:307-331.

The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. In particular, BLASTX and TBLASTN are convenient methods to compare degenerate sequences. See, Current Protocols in Molecular Biology, Chapter 19, Ausubel et al., Eds., Greene Publishing and Wiley-Interscience, New York (1995); Altschul et al. (1990) J. Mol. Biol. 215:403-410; and Altschul et al. (1997) Nucl Acids Res. 25:3389-3402.

Software for performing BLAST analyses is publicly available, e.g., through the National Center for Biotechnology Information (NCBI). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) PNAS (USA) 89:10915).

In addition to calculating percent sequence identity, the BLAST algorithm also performs a statistical analysis of the similarity between two sequences (see, e.g., Karlin & Altschul (1993) PNAS (USA) 90:5873-5877). One measure of similarity provided by the BLAST algorithm is the smallest sum probability (P(N)), which provides an indication of the probability by which a match between two nucleotide or amino acid sequences would occur by chance.

BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen (1993) Comput. Chem. 17:149-163) and XNU (Clayerie and States, Comput. Chem. (1993) 17:191-201) low-complexity filters can be employed alone or in combination.

Unless otherwise stated, nucleotide and protein identity/similarity values provided herein are calculated using the GAP algorithm (GCG Version 10) under default values. GAP (Global Alignment Program) can also be used to compare a polynucleotide or polypeptide of the present invention with a reference sequence. GAP uses the algorithm of Needleman and Wunsch (J. Mol. Biol. 48:443-453, 1970) to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can each independently be: 0, 1, 2, 3, 4, 5, 6, 7, 8, 9, 10, 15, 20, 30, 40, 50, 60 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. Version 10 of the Wisconsin Genetics Software Package uses the scoring matrix BLOSUM62 for polypeptide comparisons (Henikoff & Henikoff (1989) PNAS (USA) 89:10915) and nwsgapdna.cmp for polynucleotide comparisons.

Multiple alignment of the sequences can be performed using the CLUSTAL method of alignment (Higgins and Sharp (1989) CABIOS. 5:151-153) with the default parameters (GAP PENALTY=10, GAP LENGTH PENALTY=10). Default parameters for pairwise alignments using the CLUSTAL method are KTUPLE 1, GAP PENALTY=3, WINDOW=5 and DIAGONALS SAVED=5.

(c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller (1988) Comp. Appl. Biol. Sci. 4:11-17, e.g., as implemented in the program PC/GENE (Intelligenetics, Mountain View, Calif., USA).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

Utilities

The present invention provides, among other things, compositions and methods for modulating (i.e., increasing or decreasing) the level of RAD30/Polη polynucleotides and polypeptides, and therefore translesion DNA synthesis, in plants. In particular, the RAD30/Polη polynucleotides and polypeptides of the present invention can be expressed temporally or spatially, e.g., at developmental stages, in tissues, and/or in quantities, which are uncharacteristic of non-recombinantly engineered plants. Thus, the present invention provides utility in such exemplary applications as in the regulation of DNA repair and targeted gene modifications. In regard to targeted modifications, the present invention can be used to modify any sequence, including but not limited to polypeptide coding regions, UTR's, promoters, enhancers or other regulators of gene expression. The types of site-directed modifications to a nucleotide sequence include any changes which could suppress gene expression, such as the introduction of a premature stop codon, frameshift mutation or changes to a promoter or other UTR, and the like, or increase gene expression or protein activity such as alteration of codons, or alterations to UTR's and the like.

The RAD30/Polη DNA repair pathway involves accurate and efficient de novo synthesis of DNA using the damaged DNA as a template, called translesion synthesis (TLS). Modulation of RAD30/Polη levels could be used to regulate DNA repair. Some transformation methods may damage the DNA of the target cells, increased expression of RAD30/Polη may increase the efficiency of DNA repair and therefore increase transformation efficiency.

Overexpression of RAD30/Polη may lead to increased DNA repair and therefore increased tolerance or resistance to environmental mutagens. Further, overexpression of RAD30/Polη may enhance the ability to specifically engineer plants with enhanced or compromised tolerance to a stressful environment. In turn, these modified plants could be used as a biological assay for gene targeting restoration of wild type phenotype. For example, a point mutation of G to A in the heat shock protein HSP101 (Genbank accession U13949) converts E637 (GAA) to a lysine residue (AAA) and produces hot1 mutants with greatly reduced thermotolerance, which can be assayed by hypocotyl elongation (Hong, S-K and Vierling, E (2000) PNAS (USA) 97:43924397). In another example, molybdenum is a necessary cofactor in the carbon, nitrogen and sulfur cycles. Creating a G to A point mutation in the molybdenum cofactor biosynthetic protein Cnx1 (Genbank accession L47323) changes G108 to an aspartate residue resulting in a cnx1 mutant that cannot assimilate nitrogen. Grown in culture on reduced nitrogen, the mutants shown a retarded phenotype (Schwartz, G et al. (2000) Plant Cell 12:2455-2472).

Suppression of RAD30/Polη may provide a method to increase the efficiency of mutagenesis, to provide more genetic diversity in a population, to generate a mutagenized population for gene identification, phenotypic selection or for use as a model system for the screening, detection and/or study of putative toxins.

Introduction of RAD30/Polη along with a modification template for a target polynucleotide sequence of interest could be used to produce heritable, specific nucleotide sequence changes to the target gene. In some embodiments this method could be used, for example, to regulate herbicide, disease or insect resistance genes, male sterility genes, biosynthetic pathway or regulatory genes by targeting the change to either a regulatory element, such as a promoter or terminator, or by targeting the change to the polypeptide coding region of the target gene.

The present invention also provides isolated nucleic acids comprising polynucleotides of sufficient length and complementarity to a polynucleotide of the present invention to use as probes or amplification primers in the detection, quantitation, or isolation of gene transcripts. For example, isolated nucleic acids of the present invention can be used as probes in detecting deficiencies in the level of mRNA in screenings for desired transgenic plants, for detecting mutations in the gene (e.g., substitutions, deletions, or additions), for monitoring upregulation of expression or changes in enzyme activity in screening assays of compounds, for detection of any number of allelic variants (polymorphisms), orthologs, or paralogs of the gene, or for site directed mutagenesis in eukaryotic cells (see, e.g., U.S. Pat. No. 5,565,350). The isolated nucleic acids of the present invention can also be used for recombinant expression of their encoded polypeptides, or for use as immunogens in the preparation and/or screening of antibodies. The isolated nucleic acids of the present invention can also be employed for use in sense or antisense suppression of one or more genes of the present invention in a host cell, tissue, or plant. Attachment of chemical agents which bind, intercalate, cleave and/or crosslink to the isolated nucleic acids of the present invention can also be used to modulate transcription or translation.

The present invention also provides isolated proteins comprising a polypeptide (e.g., preproenzyme, proenzyme, or enzymes). The present invention also provides proteins comprising at least one epitope from a polypeptide of the present invention. The proteins of the present invention can be employed in assays for enzyme agonists or antagonists of enzyme function, or for use as immunogens or antigens to obtain antibodies specifically immunoreactive with a protein of the present invention. Such antibodies can be used in assays for expression levels, for identifying and/or isolating nucleic acids of the present invention from expression libraries, for identification of homologous polypeptides from other species, or for purification of polypeptides of the present invention.

The isolated nucleic acids and polypeptides of the present invention can be used over a broad range of plant types, for example monocots such as the species of the family Gramineae including Hordeum, Secale, Oryza, Triticum, Sorghum (e.g., S. bicolor) and Zea (e.g., Z. mays), and dicots such as Glycine.

The isolated nucleic acid and proteins of the present invention can also be used in species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browallia, Pisum, Phaseolus, Lolium, and Avena.

Nucleic Acids

The RAD30/Polη gene encodes a protein involved in DNA lesion repair. This DNA repair pathway involves accurate de novo synthesis of DNA using the damaged DNA as a template, also called translesion synthesis. As such, it is expected that regulation of RAD30/Polη will have useful application to modulate DNA repair including introduction of specific targeted gene modifications.

The present invention provides, among other things, isolated nucleic acids of RNA, DNA, and analogs and/or chimeras thereof, comprising a polynucleotide of the present invention.

A polynucleotide of the present invention is inclusive of:

(a) a polynucleotide encoding a polypeptide of SEQ ID NO: 2 including exemplary polynucleotides of SEQ ID NO: 1.

(b) a polynucleotide which is the product of amplification from a Zea mays nucleic acid library using primer pairs which selectively hybridize under stringent conditions to loci within a polynucleotide selected from the polynucleotide of SEQ ID NO: 1.

(c) a polynucleotide which selectively hybridizes to a polynucleotide of (a) or (b);

(d) a polynucleotide having a specified sequence identity with polynucleotides of (a), (b), or (c);

(e) a polynucleotide encoding a protein having a specified number of contiguous amino acids from a prototype polypeptide, wherein the protein is specifically recognized by antisera elicited by presentation of the protein and wherein the protein does not detectably immunoreact to antisera which has been fully immunosorbed with the protein;

(f) complementary sequences of polynucleotides of (a), (b), (c), (d), or (e);

(g) a polynucleotide comprising at least a specific number of contiguous nucleotides from a polynucleotide of (a), (b), (c), (d), (e), or (f);

(h) an isolated polynucleotide from a full-length enriched cDNA library having the physico-chemical property of selectively hybridizing to a polynucleotide of (a), (b), (c), (d), (e), (f), or (g); and

(i) an isolated polynucleotide made by the process of: 1) providing a full-length enriched nucleic acid library, 2) selectively hybridizing the polynucleotide to a polynucleotide of (a), (b), (c), (d), (e), (f), (g), or (h), thereby isolating the polynucleotide from the nucleic acid library.

A. Polynucleotides Encoding a Polypeptide of the Present Invention

As indicated in (a), above, the present invention provides isolated nucleic acids comprising a polynucleotide of the present invention, wherein the polynucleotide encodes a polypeptide of the present invention. Every nucleic acid sequence herein that encodes a polypeptide also, by reference to the genetic code, describes every possible silent variation of the nucleic acid. These sequences include degenerate sequences. One of ordinary skill will recognize that each codon in a nucleic acid (except AUG, which is ordinarily the only codon for methionine; and UGG, which is ordinarily the only codon for tryptophan) can be modified to yield a functionally identical molecule. Thus, each silent variation of a nucleic acid which encodes a polypeptide of the present invention is implicit in each described polypeptide sequence and is within the scope of the present invention. Accordingly, the present invention includes polynucleotides of SEQ ID NO: 1, and polynucleotides encoding a polypeptide of SEQ ID NO: 2.

B. Polynucleotides Amplified from a Plant Nucleic Acid Library

As indicated in (b), above, the present invention provides an isolated nucleic acid comprising a polynucleotide of the present invention, wherein the polynucleotides are amplified, under nucleic acid amplification conditions, from a plant nucleic acid library. Nucleic acid amplification conditions for each of the variety of amplification methods are well known to those of ordinary skill in the art. The plant nucleic acid library can be constructed from a monocot such as a cereal crop. Exemplary cereals include corn, sorghum, oat, barley, wheat, or rice. The plant nucleic acid library can also be constructed from a dicot such as soybean, sunflower, safflower, alfalfa, or canola. Zea mays lines B73, PHRE1, A632, BMS-P2#10, W23, and Mo17 are known and publicly available. Other publicly known and available maize lines can be obtained from the Maize Genetics Cooperation (Urbana, Ill.). Wheat lines are available from the Wheat Genetics Resource Center (Manhattan, Kans.).

The nucleic acid library may be a cDNA library, a genomic library, or a library generally constructed from nuclear transcripts at any stage of intron processing. cDNA libraries can be normalized to increase the representation of relatively rare cDNAs. In optional embodiments, the cDNA library is constructed using an enriched full-length cDNA synthesis method. Examples of such methods include Oligo-Capping (Maruyama, K. and Sugano, S. (1994) Gene 138:171-174), Biotinylated CAP Trapper (Carninci et al. (1996) Genomics 37:327-336), and CAP Retention Procedure (Edery, E. et al. (1995) Mol Cell Biol 15:3363-3371). Rapidly growing tissues or rapidly dividing cells are preferred for use as an mRNA source for construction of a cDNA library. Growth stages of corn is described in “How a Corn Plant Develops,” Special Report No. 48, Iowa State University of Science and Technology Cooperative Extension Service, Ames, Iowa, reprinted February 1993.

A polynucleotide of this embodiment (or subsequences thereof can be obtained, for example, by using amplification primers which are selectively hybridized and primer extended, under nucleic acid amplification conditions, to at least two sites within a polynucleotide of the present invention, or to two sites within the nucleic acid which flank and comprise a polynucleotide of the present invention, or to a site within a polynucleotide of the present invention and a site within the nucleic acid which comprises it. Methods for obtaining 5′ and/or 3′ ends of a vector insert are well known in the art. See, e.g., RACE (Rapid Amplification of Complementary Ends) as described in Frohman, Mass. in PCR Protocols: A Guide to Methods and Applications (1990) MA Innis et al. Eds., Academic Press, Inc., San Diego, pp. 28-38; U.S. Pat. No. 5,470,722; Current Protocols in Molecular Biology, Unit 15.6, Ausubel et al., Eds., Greene Publishing and Wiley-Interscience, New York (1995); and Frohman and Martin (1989) Techniques 1:165.

Optionally, the primers are complementary to a subsequence of the target nucleic acid which they amplify but may have a sequence identity ranging from about 85% to 99% relative to the polynucleotide sequence which they are designed to anneal to. As those skilled in the art will appreciate, the sites to which the primer pairs will selectively hybridize are chosen such that a single contiguous nucleic acid can be formed under the desired nucleic acid amplification conditions. The primer length in nucleotides is selected from the group of integers consisting of from at least 15 to 50. Thus, the primers can be at least 15, 18, 20, 25, 30, 40, or 50 nucleotides in length. Those of skill will recognize that a lengthened primer sequence can be employed to increase specificity of binding (i.e., annealing) to a target sequence. A non-annealing sequence at the 5′end of a primer (a “tail”) can be added, for example, to introduce a cloning site at the terminal ends of the amplicon.

The amplification products can be translated using expression systems well known to those of skill in the art. The resulting translation products can be confirmed as polypeptides of the present invention by, for example, assaying for the appropriate catalytic activity (e.g., specific activity and/or substrate specificity), or verifying the presence of one or more epitopes which are specific to a polypeptide of the present invention. Methods for protein synthesis from PCR derived templates are known in the art and available commercially. See, e.g., Amersham Life Sciences, Inc, Catalog '97, p.354.

C. Polynucleotides Which Selectively Hybridize to a Polynucleotide of (A) or (B)

As indicated in (c), above, the present invention provides isolated nucleic acids comprising polynucleotides of the present invention, wherein the polynucleotides selectively hybridize, under selective hybridization conditions, to a polynucleotide of sections (A) or (B) as discussed above. Thus, the polynucleotides of this embodiment can be used for isolating, detecting, and/or quantifying nucleic acids comprising the polynucleotides of (A) or (B). For example, polynucleotides of the present invention can be used to identify, isolate, or amplify partial or full-length clones in a deposited library. In some embodiments, the polynucleotides are genomic or cDNA sequences isolated or otherwise complementary to a cDNA from a dicot or monocot nucleic acid library. Exemplary species of monocots and dicots include, but are not limited to: maize, canola, soybean, cotton, wheat, sorghum, sunflower, alfalfa, oats, sugar cane, millet, barley, and rice. The cDNA library comprises at least 50% to 95% full-length sequences (for example, at least 50%, 60%, 70%, 80%, 90%, or 95% full-length sequences). The cDNA libraries can be normalized to increase the representation of rare sequences. See, e.g., U.S. Pat. No. 5,482,845. Low stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% to 80% sequence identity and can be employed to identify orthologous or paralogous sequences.

D. Polynucleotides Having a Specific Sequence Identity with the Polynucleotides of (A), (B) or (C)

As indicated in (d), above, the present invention provides isolated nucleic acids comprising polynucleotides of the present invention, wherein the polynucleotides have a specified identity at the nucleotide level to a polynucleotide as disclosed above in sections (A), (B), or (C), above. Identity can be calculated using, for example, the BLAST, CLUSTALW, or GAP algorithms under default conditions. The percentage of identity to a reference sequence is at least 50% and, rounded upwards to the nearest integer, can be expressed as an integer selected from the group of integers consisting of from 50 to 99. Thus, for example, the percentage of identity to a reference sequence can be at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%.

These polynucleotides of this embodiment can also be evaluated by comparison of the percent sequence identity shared by the polypeptides they encode. For example, isolated nucleic acids which encode a polypeptide with a given percent sequence identity to the polypeptide of SEQ ID NO: 2 are disclosed. Identity can be calculated using, for example, the BLAST, CLUSTALW, or GAP algorithms under default conditions. The percentage of identity to a reference sequence is at least 50% and, rounded upwards to the nearest integer, can be expressed as an integer selected from the group of integers consisting of from 50 to 99. Thus, for example, the percentage of identity to a reference sequence can be at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%.

The polynucleotides of this embodiment will encode a polypeptide that will share an epitope with a polypeptide encoded by the polynucleotides of sections (A), (B), or (C). Thus, these polynucleotides encode a first polypeptide which elicits production of antisera comprising antibodies which are specifically reactive to a second polypeptide encoded by a polynucleotide of (A), (B), or (C). However, the first polypeptide does not bind to antisera raised against itself when the antisera has been fully immunosorbed with the first polypeptide. Hence, the polynucleotides of this embodiment can be used to generate antibodies for use in, for example, the screening of expression libraries for nucleic acids comprising polynucleotides of (A), (B), or (C), or for purification of, or in immunoassays for, polypeptides encoded by the polynucleotides of (A), (B), or (C). The polynucleotides of this embodiment comprise nucleic acid sequences which can be employed for selective hybridization to a polynucleotide encoding a polypeptide of the present invention.

Screening polypeptides for specific binding to antisera can be conveniently achieved using peptide display libraries. This method involves the screening of large collections of peptides for individual members having the desired function or structure. Antibody screening of peptide display libraries is well known in the art. The displayed peptide sequences can be from 3 to 5000 or more amino acids in length, frequently from 5-100 amino acids long, and often from about 8 to 15 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT patent publication Nos. 91/17271, 91/18980, 91/19818, and 93/08278. Other systems for generating libraries of peptides have aspects of both in vitro chemical synthesis and recombinant methods. See, PCT Patent publication Nos. 92/05258, 92/14843, and 97/20078. See also, U.S. Pat. Nos. 5,658,754; and 5,643,768. Peptide display libraries, vectors, and screening kits are commercially available from such suppliers as Invitrogen (Carlsbad, Calif.).

E. Polynucleotides Encoding a Protein Having a Subsequence from a Prototype Polypeptide and Cross-Reactive to the Prototype Polypeptide

As indicated in (e), above, the present invention provides isolated nucleic acids comprising polynucleotides of the present invention, wherein the polynucleotides encode a protein having a subsequence of contiguous amino acids from a prototype polypeptide of the present invention such as are provided in (a), above. The length of contiguous amino acids from the prototype polypeptide is selected from the group of integers consisting of from at least 10 to the number of amino acids within the prototype sequence. Thus, for example, the polynucleotide can encode a polypeptide having a subsequence having at least 10, 15, 20, 25, 30, 35, 40, 45, 50, 55, or 60, contiguous amino acids from the prototype polypeptide. Further, the number of such subsequences encoded by a polynucleotide of the instant embodiment can be any integer selected from the group consisting of from 1 to 20, such as 2, 3, 4, or 5. The subsequences can be separated by any integer of nucleotides from 1 to the number of nucleotides in the sequence such as at least 5, 10, 15, 25, 50, 100, or 200 nucleotides.

The proteins encoded by polynucleotides of this embodiment, when presented as an immunogen, elicit the production of polyclonal antibodies which specifically bind to a prototype polypeptide such as but not limited to, a polypeptide encoded by the polynucleotide of (a) or (b), above. Generally, however, a protein encoded by a polynucleotide of this embodiment does not bind to antisera raised against the prototype polypeptide when the antisera has been fully immunosorbed with the prototype polypeptide. Methods of making and assaying for antibody binding specificity/affinity are well known in the art. Exemplary immunoassay formats include ELISA, competitive immunoassays, radioimmunoassays, Western blots, indirect immunofluorescent assays and the like.

In one assay method, fully immunosorbed and pooled antisera which is elicited to the prototype polypeptide can be used in a competitive binding assay to test the protein. The concentration of the prototype polypeptide required to inhibit 50% of the binding of the antisera to the prototype polypeptide is determined. If the amount of the protein required to inhibit binding is less than twice the amount of the prototype protein, then the protein is said to specifically bind to the antisera elicited to the immunogen. Accordingly, the proteins of the present invention embrace allelic variants, conservatively modified variants, and minor recombinant modifications to a prototype polypeptide.

A polynucleotide of the present invention optionally encodes a protein having a molecular weight as the non-glycosylated protein within 20% of the molecular weight of the full-length non-glycosylated polypeptides of the present invention. Molecular weight can be readily determined by SDS-PAGE under reducing conditions. Optionally, the molecular weight is within 15% of a full length polypeptide of the present invention, or within at least 10% to 5%, or 3%, 2%, or 1% of a full length polypeptide of the present invention.

Optionally, the polynucleotides of this embodiment will encode a protein having a specific enzymatic activity at least 50%, 60%, 80%, or 90% of a cellular extract comprising the native, endogenous full-length polypeptide of the present invention. Further, the proteins encoded by polynucleotides of this embodiment will optionally have a substantially similar affinity constant (Kₘ) and/or catalytic activity (i.e., the microscopic rate constant, k_{cat}) as the native endogenous, full-length protein. Those of skill in the art will recognize that k_{cat}/Kₘ value determines the specificity for competing substrates and is often referred to as the specificity constant. Proteins of this embodiment can have a k_{cat}/Kₘ value at least 10% of a full-length polypeptide of the present invention as determined using the endogenous substrate of that polypeptide. Optionally, the k_{cat}/Kₘ value will be at least 20%, 30%, 40%, 50%, or at least 60%, 70%, 80%, 90%, or 95% the k_{cat}/Kₘ value of the full-length polypeptide of the present invention. Determination of k_{cat}, Kₘ, and k_{cat}/Kₘ can be determined by any number of means well known to those of skill in the art. For example, the initial rates (i.e., the first 5% or less of the reaction) can be determined using rapid mixing and sampling techniques (e.g., continuous-flow, stopped-flow, or rapid quenching techniques), flash photolysis, or relaxation methods (e.g., temperature jumps) in conjunction with such exemplary methods of measuring as spectrophotometry, spectrofluorimetry, nuclear magnetic resonance, or radioactive procedures. Kinetic values are conveniently obtained using a Lineweaver-Burk or Eadie-Hofstee plot.

F. Polynucleotides Complementary to the Polynucleotides of (A)-(E)

As indicated in (f), above, the present invention provides isolated nucleic acids comprising polynucleotides complementary to the polynucleotides of paragraphs A-E, above. As those of skill in the art will recognize, complementary sequences base-pair throughout the entirety of their length with the polynucleotides of sections (A)-(E) (i.e., have 100% sequence identity over their entire length). Complementary bases associate through hydrogen bonding in double stranded nucleic acids. For example, the following base pairs are complementary: guanine and cytosine; adenine and thymine; and adenine and uracil.

G. Polynucleotides Which are Subsequences of the Polynucleotides of (A)-(F)

As indicated in (g), above, the present invention provides isolated nucleic acids comprising polynucleotides which comprise at least 15 contiguous bases from the polynucleotides of sections (A) through (F) as discussed above. The length of the polynucleotide is given as an integer selected from the group consisting of from at least 15 to the length of the nucleic acid sequence from which the polynucleotide is a subsequence of. Thus, for example, polynucleotides of the present invention are inclusive of polynucleotides comprising at least 15, 20, 25, 30, 35, 40, 45, 50, 55, 60, 65, 70, 75, 80, 90, 100 or 200 contiguous nucleotides in length from the polynucleotides of (A)-(F). Optionally, the number of such subsequences encoded by a polynucleotide of the instant embodiment can be any integer selected from the group consisting of from 1 to 20, such as 2, 3, 4, or 5. The subsequences can be separated by any integer of nucleotides from 1 to the number of nucleotides in the sequence such as at least 5, 10, 15, 25, 50, 100, or 200 nucleotides.

Subsequences can be made by in vitro synthetic, in vitro biosynthetic, or in vivo recombinant methods. In optional embodiments, subsequences can be made by nucleic acid amplification. For example, nucleic acid primers will be constructed to selectively hybridize to a sequence (or its complement) within, or co-extensive with, the coding region.

The subsequences of the present invention can comprise structural characteristics of the sequence from which it is derived. Alternatively, the subsequences can lack certain structural characteristics of the larger sequence from which it is derived such as a poly (A) tail. Optionally, a subsequence from a polynucleotide encoding a polypeptide having at least one epitope in common with a prototype polypeptide sequence as provided in (a), above, may encode an epitope in common with the prototype sequence. Alternatively, the subsequence may not encode an epitope in common with the prototype sequence but can be used to isolate the larger sequence by, for example, nucleic acid hybridization with the sequence from which it's derived. Subsequences can be used to modulate or detect gene expression by introducing into the subsequences compounds which bind, intercalate, cleave and/or crosslink to nucleic acids. Exemplary compounds include acridine, psoralen, phenanthroline, naphthoquinone, daunomycin or chloroethylaminoaryl conjugates.

H. Polynucleotides From a Full-length Enriched cDNA Library Having the Physico-Chemical Property of Selectively Hybridizing to a Polynucleotide of (A)-(G)

As indicated in (h), above, the present invention provides an isolated polynucleotide from a full-length enriched cDNA library having the physico-chemical property of selectively hybridizing to a polynucleotide of paragraphs (A), (B), (C), (D), (E), (F), or (G) as discussed above. Methods of constructing full-length enriched cDNA libraries are known in the art and discussed briefly below. The cDNA library comprises at least 50% to 95% full-length sequences (for example, at least 50%, 60%, 70%, 80%, 90%, or 95% full-length sequences). The cDNA library can be constructed from a variety of tissues from a monocot or dicot at a variety of developmental stages. Exemplary species include maize, wheat, canola, soybean, cotton, sorghum, sunflower, alfalfa, oats, sugar cane, millet, barley, and rice. Methods of selectively hybridizing, under selective hybridization conditions, a polynucleotide from a full-length enriched library to a polynucleotide of the present invention are known to those of ordinary skill in the art. Any number of stringency conditions can be employed to allow for selective hybridization. In optional embodiments, the stringency allows for selective hybridization of sequences having at least 70%, 75%, 80%, 85%, 90%, 95%, or 98% sequence identity over the length of the hybridized region. Full-length enriched cDNA libraries can be normalized to increase the representation of rare sequences.

I. Polynucleotide Products Made by a cDNA Isolation Process

As indicated in (i), above, the present invention provides an isolated polynucleotide made by the process of: 1) providing a full-length enriched nucleic acid library, 2) selectively hybridizing the polynucleotide to a polynucleotide of paragraphs (A), (B), (C), (D), (E), (F), (G), or (H) as discussed above, and thereby isolating the polynucleotide from the nucleic acid library. Full-length enriched nucleic acid libraries are constructed as discussed in paragraph (H) and below. Selective hybridization conditions are as discussed in paragraph (C). Nucleic acid purification procedures are well known in the art. Purification can be conveniently accomplished using solid-phase methods; such methods are well known to those of skill in the art and kits are available from commercial suppliers such as Advanced Biotechnologies (Surrey, UK). For example, a polynucleotide of paragraphs (A)-(H) can be immobilized to a solid support such as a membrane, bead, or particle. See, e.g., U.S. Pat. No. 5,667,976. The polynucleotide product of the present process is selectively hybridized to an immobilized polynucleotide and the solid support is subsequently isolated from non-hybridized polynucleotides by methods including, but not limited to, centrifugation, magnetic separation, filtration, electrophoresis, and the like.

Construction of Nucleic Acids

The isolated nucleic acids of the present invention can be made using (a) standard recombinant methods, (b) synthetic techniques, or combinations thereof. In some embodiments, the polynucleotides of the present invention will be cloned, amplified, or otherwise constructed from a monocot such as corn, rice, or wheat, or a dicot such as soybean.

The nucleic acids may conveniently comprise sequences in addition to a polynucleotide of the present invention. For example, a multi-cloning site comprising one or more endonuclease restriction sites may be inserted into the nucleic acid to aid in isolation of the polynucleotide. Also, translatable sequences may be inserted to aid in the isolation of the translated polynucleotide of the present invention. For example, a hexa-histidine marker sequence provides a convenient means to purify the proteins of the present invention. A polynucleotide of the present invention can be attached to a vector, adapter, or linker for cloning and/or expression of a polynucleotide of the present invention. Additional sequences may be added to such cloning and/or expression sequences to optimize their function in cloning and/or expression, to aid in isolation of the polynucleotide, or to improve the introduction of the polynucleotide into a cell. Typically, the length of a nucleic acid of the present invention less the length of its polynucleotide of the present invention is less than 20 kilobase pairs, often less than 15 kb, and frequently less than 10 kb. Use of cloning vectors, expression vectors, adapters, and linkers is well known and extensively described in the art. For a description of various nucleic acids see, for example, Stratagene Cloning Systems, Catalogs 1999 (La Jolla, Calif.); and, Amersham Life Sciences, Inc, Catalog '99 (Arlington Heights, Ill.).

A. Recombinant Methods for Constructing Nucleic Acids

The isolated nucleic acid compositions of this invention, such as RNA, cDNA, genomic DNA, or a hybrid thereof, can be obtained from plant biological sources using any number of cloning methodologies known to those of skill in the art. In some embodiments, oligonucleotide probes which selectively hybridize, under stringent conditions, to the polynucleotides of the present invention are used to identify the desired sequence in a cDNA or genomic DNA library. Isolation of RNA, and construction of cDNA and genomic libraries is well known to those of ordinary skill in the art. See, e.g., Plant Molecular Biology; A Laboratory Manual, Clark, Ed., Springer-Verlag, Berlin (1997); and, Current Protocols in Molecular Biology, Ausubel et al., Eds., Greene Publishing and Wiley-Interscience, New York (1995).

A1. Construction of a cDNA Library

Construction of a cDNA library generally entails five steps. First, first strand cDNA synthesis is initiated from a poly(A)⁺ mRNA template using a poly(dT) primer or random hexanucleotides. Second, the resultant RNA-DNA hybrid is converted into double stranded cDNA, typically by reaction with a combination of RNAse H and DNA polymerase I (or Klenow fragment). Third, the termini of the double stranded cDNA are ligated to adaptors. Ligation of the adaptors can produce cohesive ends for cloning. Fourth, size selection of the double stranded cDNA eliminates excess adaptors and primer fragments, and eliminates partial cDNA molecules due to degradation of mRNAs or the failure of reverse transcriptase to synthesize complete first strands. Fifth, the cDNAs are ligated into cloning vectors and packaged. cDNA synthesis protocols are well known to the skilled artisan and are described in such standard references as: Plant Molecular Biology: A Laboratory Manual, Clark, Ed., Springer-Verlag, Berlin (1997); and Current Protocols in Molecular Biology, Ausubel et al., Eds., Greene Publishing and Wiley-Interscience, New York (1995). cDNA synthesis kits are available from a variety of commercial vendors such as Stratagene or Pharmacia.

A2. Full-length Enriched cDNA Libraries

A number of cDNA synthesis protocols have been described which provide enriched full-length cDNA libraries. Enriched full-length cDNA libraries are constructed to comprise at least 60%, or at least 70%, 80%, 90% or 95% full-length inserts amongst clones containing inserts. The length of insert in such libraries can be at least 2, 3, 4, 5, 6, 7, 8, 9, 10 or more kilobase pairs. Vectors to accommodate inserts of these sizes are known in the art and available commercially. See, e.g., Stratagene lambda ZAP Express (cDNA cloning vector with 0 to 12 kb cloning capacity). An exemplary method of constructing a greater than 95% pure full-length cDNA library is described by Carninci et al. (1996) Genomics 37:327-336. Other methods for producing full-length libraries are known in the art. See, e.g., Edery et al. (1995) Mol. Cell Biol. 15(6):3363-3371; and PCT Application WO 96/34981.

A3. Normalized or Subtracted cDNA Libraries

A non-normalized cDNA library represents the mRNA population of the tissue from which it was made. Since unique clones are out-numbered by clones derived from highly expressed genes their isolation can be laborious. Normalization of a cDNA library is the process of creating a library in which each clone is more equally represented. Construction of normalized libraries is described in Ko (1990) Nucl. Acids Res. 18(19):5705-5711; Patanjali et al. (1991) PNAS (USA) 88:1943-1947; U.S. Pat. Nos. 5,482,685, 5,482,845, and 5,637,685. In an exemplary method described by Soares et al. (1994) PNAS (USA) 91:9228-9232, normalization resulted in reduction of the abundance of clones from a range of four orders of magnitude to a narrow range of only 1 order of magnitude.

Subtracted cDNA libraries are another means to increase the proportion of less abundant cDNA species. In this procedure, cDNA prepared from one pool of mRNA is depleted of sequences present in a second pool of mRNA by hybridization. The cDNA:mRNA hybrids are removed and the remaining un-hybridized cDNA pool is enriched for sequences unique to that pool. See, Foote et al. in Plant Molecular Biology: A Laboratory Manual, Clark, Ed., Springer-Verlag, Berlin (1997); Kho and Zarbl (1991) Technique 3(2):58-63; Sive and St. John (1988) Nucl. Acids Res. 16(22):10937; Current Protocols in Molecular Biology, Ausubel et al., Eds., Greene Publishing and Wiley-Interscience, New York (1995); and Swaroop et al. (1991) Nucl. Acids Res. 19(8):1954. cDNA subtraction kits are commercially available. See, e.g., PCR-Select (Clontech, Palo Alto, Calif.).

A4. Construction of a Genomic Library

To construct genomic libraries, large segments of genomic DNA are generated by fragmentation, e.g. using restriction endonucleases, and are ligated with vector DNA to form concatemers that can be packaged into the appropriate vector. Methodologies to accomplish these ends, and sequencing methods to verify the sequence of nucleic acids are well known in the art. Examples of appropriate molecular biological techniques and instructions sufficient to direct persons of skill through many construction, cloning, and screening methodologies are found in Sambrook et al., Molecular Cloning: A Laboratory Manual, 2nd Ed., Cold Spring Harbor Laboratory Vols. 1-3 (1989); Methods in Enzymology, Vol. 152: Guide to Molecular Cloning Techniques, Berger and Kimmel, Eds., San Diego: Academic Press, Inc. (1987); Current Protocols in Molecular Biology, Ausubel et al., Eds., Greene Publishing and Wiley-Interscience, New York (1995); and Plant Molecular Biology: A Laboratory Manual, Clark, Ed., Springer-Verlag, Berlin (1997). Kits for construction of genomic libraries are also commercially available.

A5. Nucleic Acid Screening and Isolation Methods

The cDNA or genomic library can be screened using a probe based upon the sequence of a polynucleotide of the present invention such as those disclosed herein. Probes may be used to hybridize with genomic DNA or cDNA sequences to isolate homologous genes in the same or different plant species. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by temperature, ionic strength, pH and the presence of a partially denaturing solvent such as formamide. For example, the stringency of hybridization is conveniently varied by changing the polarity of the reactant solution through manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100 percent; however, it should be understood that minor sequence variations in the probes and primers may be compensated for by reducing the stringency of the hybridization and/or wash medium.

The nucleic acids of interest can also be amplified from nucleic acid samples using amplification techniques. For instance, polymerase chain reaction (PCR) technology can be used to amplify the sequences of polynucleotides of the present invention and related genes directly from genomic DNA or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, Sambrook, and Ausubel (supra), as well as Mullis et al., U.S. Pat. No. 4,683,202 (1987); and, PCR Protocols A Guide to Methods and Applications, Innis et al., Eds., Academic Press Inc., San Diego, Calif. (1990). Commercially available kits for genomic PCR amplification are known in the art. See, e.g., Advantage-GC Genomic PCR Kit (Clontech). The T4 gene 32 protein (Boehringer Mannheim) can be used to improve yield of long PCR products.

PCR-based screening methods have also been described. Wilfinger et al. describe a PCR-based method in which the longest cDNA is identified in the first step so that incomplete clones can be eliminated from study. Bio Techniques (1997) 22(3):481-486. In that method, a primer pair is synthesized with one primer annealing to the 5′ end of the sense strand of the desired cDNA and the other primer to the vector. Clones are pooled to allow large-scale screening. By this procedure, the longest possible clone is identified among candidate clones. Further, the PCR product is used solely as a diagnostic for the presence of the desired cDNA and does not utilize the PCR product itself. Such methods are particularly effective in combination with a full-length cDNA construction methodology, above.

B. Synthetic Methods for Constructing Nucleic Acids

The isolated nucleic acids of the present invention can also be prepared by direct chemical synthesis by methods such as the phosphotriester method of Narang et al. (1979) Meth. Enzymol. 68:90-99; the phosphodiester method of Brown et al. (1979) Meth. Enzymol. 68:109-151; the diethylphosphoramidite method of Beaucage et al. (1981) Tetra. Lett. 22:1859-1862; the solid phase phosphoramidite triester method described by Beaucage and Caruthers (1981) Tetra. Leffs. 22(20):1859-1862, e.g., using an automated synthesizer, e.g., as described in Needham-VanDevanter et al. (1984) Nucl. Acids Res. 12:6159-6168; and the solid support method of U.S. Pat. No. 4,458,066. Chemical synthesis generally produces a single stranded oligonucleotide. This may be converted into double stranded DNA by hybridization with a complementary sequence, or by polymerization with a DNA polymerase using the single strand as a template. One of skill will recognize that while chemical synthesis of DNA is best employed for sequences of about 100 bases or less, longer sequences may be obtained by the ligation of shorter sequences.

Recombinant Expression Cassettes

The present invention further provides recombinant expression cassettes comprising a nucleic acid of the present invention. A nucleic acid sequence coding for the desired polypeptide of the present invention, for example a cDNA or a genomic sequence encoding a full length polypeptide of the present invention, can be used to construct a recombinant expression cassette which can be introduced into the desired host cell. A recombinant expression cassette will typically comprise a polynucleotide of the present invention, in either sense or antisense orientation, operably linked to transcriptional initiation regulatory sequences which will direct the transcription of the polynucleotide in the intended host cell, such as tissues of a transformed plant.

Exemplary plant promoters include, but are not limited to, those that are obtained from plants, plant viruses, and bacteria which comprise genes expressed in plant cells such Agrobacterium or Rhizobium. Examples of promoters under developmental control include promoters that preferentially initiate transcription in certain tissues, such as leaves, roots, or seeds. Such promoters are referred to as “tissue preferred”. Promoters which initiate transcription only in certain, tissue are referred to as “tissue specific”. A “cell type” specific promoter primarily drives expression in certain cell types in one or more organs, for example, vascular cells in roots or leaves. An “inducible” or “repressible” promoter is a promoter which is under environmental control. Examples of environmental conditions that may effect transcription by inducible promoters include anaerobic conditions or the presence of light. Tissue specific, tissue preferred, cell type specific, and inducible promoters constitute the class of “non-constitutive” promoters. A “constitutive” promoter is a promoter which is active under most environmental conditions.

For example, plant expression vectors may include (1) a cloned plant gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site, and/or a polyadenylation signal.

A plant promoter fragment can be employed which will direct expression of a polynucleotide of the present invention in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumefaciens, the ubiquitin 1 promoter, the Smas promoter, the cinnamyl alcohol dehydrogenase promoter (U.S. Pat. No. 5,683,439), the Nos promoter, the pEmu promoter, the rubisco promoter, and the GRP1-8 promoter. One exemplary promoter is the ubiquitin promoter, which can be used to drive expression of the present invention in maize embryos or embryogenic callus.

Alternatively, the plant promoter can direct expression of a polynucleotide of the present invention in a specific tissue or may be otherwise under more precise environmental or developmental control. Such promoters are referred to here as “inducible” promoters. Environmental conditions that may effect transcription by inducible promoters include pathogen attack, anaerobic conditions, or the presence of light. Examples of inducible promoters are the Adh1 promoter which is inducible by hypoxia or cold stress, the Hsp70 promoter which is inducible by heat stress, and the PPDK promoter which is inducible by light.

Examples of promoters under developmental control include promoters that initiate transcription only, or preferentially, in certain tissues, such as leaves, roots, fruit, seeds, or flowers. Exemplary promoters include the anther specific promoter 5126 (U.S. Pat. Nos. 5,689,049 and 5,689,051), and seed specific promoters such as the glob-1 promoter, and the gamma-zein promoter. The operation of a promoter may also vary depending on its location in the genome. Thus, an inducible promoter may become fully or partially constitutive in certain locations.

For example, in order to generate a male sterile phenotype, exemplary promoters include the anther-specific promoter 5126 (supra), the tapetum-specific promoter Osg6B from rice (Yokoi, S. et al. (1997) Plant Cell Reports 16(6):363-367), the anther-specific promoter apg (Twell, D. et al. (1993) Sexual Plant Reproduction 6(4):217-224), and the anther-specific promoter fragments chiA P-A2 and chiB P-B (Van Tunen, A J et al. (1990) Plant Cell 2(5):393-402).

Both heterologous and non-heterologous (i.e., endogenous) promoters can be employed to direct expression of the nucleic acids of the present invention. These promoters can also be used, for example, in recombinant expression cassettes to drive expression of sense or antisense nucleic acids to reduce, increase, or alter concentration and/or composition of the proteins of the present invention in a desired tissue. Thus, in some embodiments, the nucleic acid construct will comprise a promoter, functional in a plant cell, operably linked to a polynucleotide of the present invention. Promoters useful in these embodiments include the endogenous promoters driving expression of a polypeptide of the present invention.

In some embodiments, isolated nucleic acids which serve as promoter or enhancer elements can be introduced in the appropriate position (generally upstream) of a non-heterologous form of a polynucleotide of the present invention so as to up or down regulate expression of a polynucleotide of the present invention. For example, endogenous promoters can be altered in vivo by mutation, deletion, and/or substitution (see, Kmiec, U.S. Pat. No. 5,565,350; Zarling et al., PCT/US93/03868), or isolated promoters can be introduced into a plant cell in the proper orientation and distance from a cognate gene of a polynucleotide of the present invention so as to control the expression of the gene. Gene expression can be modulated under conditions suitable for plant growth so as to alter the total concentration and/or alter the composition of the polypeptides of the present invention in plant cell. Thus, the present invention provides compositions, and methods for making, heterologous promoters and/or enhancers operably linked to a native, endogenous (i.e., non-heterologous) form of a polynucleotide of the present invention.

Methods for identifying promoters with a particular expression pattern, in terms of, e.g., tissue type, cell type, stage of development, and/or environmental conditions, are well known in the art. See, e.g., The Maize Handbook, Chapters 114-115, Freeling and Walbot, Eds., Springer, New York (1994); Corn and Corn Improvement, 3^{rd} edition, Chapter 6, Sprague and Dudley, Eds., American Society of Agronomy, Madison, Wis. (1988). A typical step in promoter isolation methods is identification of gene products that are expressed with some degree of specificity in the target tissue. Amongst the range of methodologies are: differential hybridization to cDNA libraries; subtractive hybridization; differential display; differential 2-D protein gel electrophoresis; DNA probe arrays; and isolation of proteins known to be expressed with some specificity in the target tissue. Such methods are well known to those of skill in the art. Commercially available products for identifying promoters are known in the art such as Clontech's (Palo Alto, Calif.) Universal GenomeWalker Kit.

For the protein-based methods, it is helpful to obtain the amino acid sequence for at least a portion of the identified protein, and then to use the protein sequence as the basis for preparing a nucleic acid that can be used as a probe to identify either genomic DNA directly, or preferably, to identify a cDNA clone from a library prepared from the target tissue. Once such a cDNA clone has been identified, that sequence can be used to identify the sequence at the 5′ end of the transcript of the indicated gene. For differential hybridization, subtractive hybridization and differential display, the nucleic acid sequence identified as enriched in the target tissue is used to identify the sequence at the 5′ end of the transcript of the indicated gene. Once such sequences are identified, starting either from protein sequences or nucleic acid sequences, any of these sequences identified as being from the gene transcript can be used to screen a genomic library prepared from the target organism. Methods for identifying and confirming the transcriptional start site are well known in the art.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added can be derived from, for example, the nopaline synthase or octopine synthase genes, or alternatively from another plant gene, or less preferably from any other eukaryotic gene.

An intron sequence can be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold. Buchman and Berg (1988) Mol. Cell Biol. 8:4395-4405; and Callis et al. (1987) Genes Dev. 1:1183-1200. Such intron enhancement of gene expression is typically greatest when placed near the 5′ end of the transcription unit. Use of maize introns Adh1-S intron 1, 2, and 6, the Bronze-1 intron are known in the art. See generally, The Maize Handbook, Chapter 116, Freeling and Walbot, Eds., Springer, New York (1994). The vector comprising the sequences from a polynucleotide of the present invention will typically comprise a marker gene which confers a selectable phenotype on plant cells. Typical vectors useful for expression of genes in higher plants are well known in the art and include vectors derived from the tumor-inducing (Ti) plasmid of Agrobacterium tumefaciens described by Rogers et al. (1987) Meth. in Enzymol. 153:253-277.

A polynucleotide of the present invention can be expressed in either sense or anti-sense orientation as desired. It will be appreciated that control of gene expression in either sense or anti-sense orientation can have a direct impact on the observable plant characteristics. Antisense technology can be conveniently used to inhibit gene expression in plants. To accomplish this, a nucleic acid segment from the desired gene is cloned and operably linked to a promoter such that the anti-sense strand of RNA will be transcribed. The construct is then transformed into plants and the antisense strand of RNA is produced. In plant cells, it has been shown that antisense RNA inhibits gene expression by preventing the accumulation of mRNA which encodes the enzyme of interest, see, e.g., Sheehy et al. (1988) PNAS (USA) 85:8805-8809; and Hiatt et al., U.S. Pat. No. 4,801,340.

Another method of suppression is sense suppression (i.e., co-suppression). Introduction of nucleic acid configured in the sense orientation has been shown to be an effective means by which to block the transcription of target genes. For an example of the use of this method to modulate expression of endogenous genes see Napoli et al. (1990) The Plant Cell 2:279-289; and U.S. Pat. No. 5,034,323.

Catalytic RNA molecules or ribozymes can also be used to inhibit expression of plant genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs. The design and use of target RNA-specific ribozymes is described in Haseloff et al. (1988) Nature 334:585-591.

A variety of cross-linking agents, alkylating agents and radical generating species as pendant groups on polynucleotides of the present invention can be used to bind, label, detect, and/or cleave nucleic acids. For example, Vlassov, VV et al. (1986) Nucl Acids Res 14:4065-4076, describe covalent bonding of a single-stranded DNA fragment with alkylating derivatives of nucleotides complementary to target sequences. A report of similar work by the same group is that by Knorre, D G et al. (1985) Biochimie 67:785-789. Iverson and Dervan also showed sequence-specific cleavage of single-stranded DNA mediated by incorporation of a modified nucleotide which was capable of activating cleavage (J Am Chem Soc (1987) 109:1241-1243). Meyer, R B et al. (1989) J Am Chem Soc 111:8517-8519, effect covalent crosslinking to a target nucleotide using an alkylating agent complementary to the single-stranded target nucleotide sequence. A photoactivated crosslinking to single-stranded oligonucleotides mediated by psoralen was disclosed by Lee, B L et al. (1988) Biochemistry 27:3197-3203. Use of crosslinking in triple-helix forming probes was also disclosed by Home et al. (1990) J Am Chem Soc 112:2435-2437. Use of N4, N4-ethanocytosine as an alkylating agent to crosslink to single-stranded oligonucleotides has also been described by Webb and Mafteucci (1986) J Am Chem Soc 108:2764-2765; (1986) Nucl Acids Res 14:7661-7674; and Feteritz et al. (1991) J. Am. Chem. Soc. 113:4000. Various compounds to bind, detect, label, and/or cleave nucleic acids are known in the art. See, for example, U.S. Pat. Nos. 5,543,507; 5,672,593; 5,484,908; 5,256,648; and, 5,681941.

Proteins

The RAD30/Polη gene encodes a protein involved in DNA lesion repair. This DNA repair pathway involves accurate de novo synthesis of DNA using the damaged DNA as a template to accurately synthesize a correct undamaged strand of DNA, also called translesion synthesis. RAD30/Polη is best characterized for its ability to accurately synthesize A-A in the proper positions using a DNA template containing a Tˆ T dimer. Enzymes involved in this pathway belong to a very large gene family, the UmuC/DinB/RAD30/Polη gene family. Members of this superfamily share important structural motifs that are critical for their TLS function. The RAD30/Polη polypeptide of the present invention contains five domains conserved from bacteria to humans as is shown in Example 6 (See also McDonald, J P et al. (1999) Genomics 60:20-30). These conserved motifs are clustered in the amino-terminal region of the protein, as is the case with other RAD30-like proteins. Motif I which extends from R12 through R30, and Motif II extending from G51 through 180 have not yet had functions assigned to them, but they are presumably critical for some aspect of RAD30/Polη function as they are conserved in prokaryotes, archaea, and eukaryotes. Motif III, amino acids E115 through L126, is conserved in all known Polη sequences (Kannouche, P et al. (2001) Genes Dev 15:158-172; and Kondratick et al. (2001) Mol Cell Biol 21:2018-2025). Motif III comprises a SIDEXX box domain involved in binding Mg++, and which may serve as the catalytic site of the enzyme. Motif IV, amino acids C206 through V232, and Motif V, amino acids V246 through L260, each contain a helix-hairpin-helix domain found in other Rad3O-like proteins, and which may be involved in DNA binding. The sequence also contains two putative nuclear localization signal sequences at positions K354-K369 and A511-K525 in the amino acid sequence. It is expected that regulation of RAD30/Polη will have useful application to modulate DNA repair in plants including introduction of specific gene targeted modifications, to create specific gene knockouts, to increase genetic diversity, or to increase transformation efficiency in plants.

The isolated proteins of the present invention comprise a polypeptide having at least 10 amino acids from a polypeptide of the present invention (or conservative variants thereof) such as those encoded by any one of the polynucleotides of the present invention as discussed more fully above. The proteins of the present invention or variants thereof can comprise any number of contiguous amino acid residues from a polypeptide of the present invention, wherein that number is selected from the group of integers consisting of from 10 to the number of residues in a full-length polypeptide of the present invention. Optionally, this subsequence of contiguous amino acids is at least 15, 20, 25, 30, 35, or 40 amino acids in length, often at least 50, 60, 70, 80, 90, 100, 125 or 150 amino acids in length. Further, the number of such subsequences can be any integer selected from the group consisting of from 1 to 20, such as 2, 3, 4, or 5.

The present invention further provides a protein comprising a polypeptide having a specified sequence identity/similarity with a polypeptide of the present invention. The percentage of sequence identity/similarity is an integer selected from the group consisting of from 50 to 99. Exemplary sequence identity/similarity values include 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, and 99%. Sequence identity can be determined using, for example, the GAP, CLUSTALW, or BLAST algorithms.

As those of skill will appreciate, the present invention includes, but is not limited to, catalytically active polypeptides of the present invention (i.e., enzymes). Catalytically active polypeptides have a specific activity of at least 20%, 30%, 40%, 50%, 60%, 70%, or at least 80%, 90%, or 95% that of the native (non-synthetic), endogenous polypeptide. Further, the substrate specificity (k_{cat}/Kₘ) is optionally substantially similar to the native, endogenous polypeptide. Typically, the Kₘ will be at least 30%, 40%, or 50%, of that of the native, endogenous polypeptide or optionally, at least 60%, 70%, 80%, or 90%. Methods of assaying and quantifying measures of enzymatic activity and substrate specificity (k_{cat}/Kₘ), are well known to those of skill in the art (see, e.g. Segel, (1976) Biochemical Calculations, 2^{nd} ed., John Wiley and Sons, New York).

Generally, the proteins of the present invention will, when presented as an immunogen, elicit production of an antibody specifically reactive to a polypeptide of the present invention. Further, the proteins of the present invention will not bind to antisera raised against a polypeptide of the present invention which has been fully immunosorbed with the same polypeptide. Immunoassays for determining binding are well known to those of skill in the art. One example of an immunoassay used to determine binding is a competitive immunoassay. Thus, the proteins of the present invention can be employed as immunogens for constructing antibodies immunoreactive to a protein of the present invention for such exemplary utilities as immunoassays or protein purification techniques.

Expression of Proteins in Host Cells

Using the nucleic acids of the present invention, one may express a protein of the present invention in a recombinantly engineered cell such as bacteria, yeast, insect, mammalian, or plant cells. The cells produce the protein in a non-natural condition (e.g., in quantity, composition, location, and/or time), because they have been genetically altered through human intervention to do so.

It is expected that those of skill in the art are knowledgeable in the numerous expression systems available for expression of a nucleic acid encoding a protein of the present invention. No attempt to describe in detail the various methods known for the expression of proteins in prokaryotes or eukaryotes will be made.

In brief summary, the expression of isolated nucleic acids encoding a protein of the present invention will typically be achieved by operably linking, for example, the DNA or cDNA to a promoter (which is either constitutive or regulatable), followed by incorporation into an expression vector. The vectors can be suitable for replication and integration in either prokaryotes or eukaryotes. Typical expression vectors contain transcription and translation terminators, initiation sequences, and promoters useful for regulation of the expression of the DNA encoding a protein of the present invention. To obtain high level expression of a cloned gene, it is desirable to construct expression vectors which contain, at the minimum, a strong promoter to direct transcription, a ribosome binding site for translational initiation, and a transcription/translation terminator. One of skill would recognize that modifications can be made to a protein of the present invention without diminishing its biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located purification sequences. Restriction sites or termination codons can also be introduced.

Synthesis of Proteins

The proteins of the present invention can be constructed using non-cellular synthetic methods. Solid phase synthesis of proteins of less than about 50 amino acids in length may be accomplished by attaching the C-terminal amino acid of the sequence to an insoluble support followed by sequential addition of the remaining amino acids in the sequence. Techniques for solid phase synthesis are described by Barany and Merrifield, Solid-Phase Peptide Synthesis, pp. 3-284 in The Peptides: Analysis, Synthesis, Biology, Vol. 2: Special Methods in Peptide Synthesis, Part A; Merrifield et al. (1963) J. Am. Chem. Soc. 85: 2149-2156; and Stewart et al. (1984) Solid Phase Peptide Synthesis, 2nd ed., Pierce Chem. Co., Rockford, Ill. Proteins of greater length may be synthesized by condensation of the amino and carboxy termini of shorter fragments. Methods of forming peptide bonds by activation of a carboxy terminal end (e.g., by the use of the coupling reagent N,N′-dicycylohexyicarbodiimide) are known to those of skill.

Purification of Proteins

The proteins of the present invention may be purified by standard techniques well known to those of skill in the art. Recombinantly produced proteins of the present invention can be directly expressed or expressed as a fusion protein. The recombinant protein may be purified by a combination of cell lysis (e.g., sonication, French press) and affinity chromatography. For fusion products, subsequent digestion of the fusion protein with an appropriate proteolytic enzyme releases the desired recombinant protein.

The proteins of this invention, recombinant or synthetic, may be purified to substantial purity by standard techniques well known in the art, including detergent solubilization, selective precipitation with such substances as ammonium sulfate, column chromatography, immunopurification methods, and others. See, for instance, R. Scopes, Protein Purification: Principles and Practice, Springer-Verlag: New York (1982); Deutscher, Guide to Protein Purification, Academic Press (1990). For example, antibodies may be raised to the proteins as described herein. Purification from E. coli can be achieved following procedures described in U.S. Pat. No. 4,511,503. The protein may then be isolated from cells expressing the protein and further purified by standard protein chemistry techniques as described herein. Detection of the expressed protein is achieved by methods known in the art and include, for example, radioimmunoassays, Western blotting techniques or immunoprecipitation.

Introduction of Nucleic Acids Into Host Cells

The method of introducing a nucleic acid of the present invention into a host cell is not critical to the instant invention. Transformation or transfection methods are conveniently used. Accordingly, a wide variety of methods have been developed to insert a DNA sequence into the genome of a host cell to obtain the transcription and/or translation of the sequence to effect phenotypic changes in the organism. Thus, any method which provides for effective introduction of a nucleic acid may be employed.

A. Plant Transformation

A nucleic acid comprising a polynucleotide of the present invention is optionally introduced into a plant. Generally, the polynucleotide will first be incorporated into a recombinant expression cassette or vector. Isolated nucleic acid acids of the present invention can be introduced into plants according to techniques known in the art. Techniques for transforming a wide variety of higher plant species are well known and described in the technical, scientific, and patent literature. Suitable methods of transforming plant cells include microinjection (Crossway et al. (1986) Biotechniques 4:320-334), electroporation (Riggs et al. (1986) PNAS (USA) 83:5602-5606), Agrobacterium mediated transformation (see for example, Zhao et al. U.S. Pat. Nos. 5,981,840; 5,563,055), direct gene transfer (Paszkowski et al. (1984) EMBO J. 3:2717-2722), and ballistic particle acceleration (see, for example, Sanford et al. U.S. Pat. No. 4,945,050; Tomes et al. “Direct DNA Transfer into Intact Plant Cells via Microprojectile Bombardment” In Gamborg and Phillips (Eds.) Plant Cell, Tissue and Organ Culture: Fundamental Methods, Springer-Verlag, Berlin (1995); and McCabe et al. (1988) Biotechnology 6:923-926). Also see, Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); Datta et al. (1990) Biotechnology 8:736-740 (rice); Klein et al. (1988) PNAS (USA) 85:4305-4309 (maize); Klein et al. (1988) Biotechnology 6:559-563 (maize); Klein et al. (1988) Plant Physiol. 91:440-444 (maize); Fromm et al. (1990) Biotechnology 8:833-839 (maize); Hooykaas-Van Slogteren & Hooykaas (1984) Nature 311:763-764; Bytebier et al. (1987) PNAS (USA) 84:5345-5349 (Liliaceae); De Wet et al. (1985) In The Experimental Manipulation of Ovule Tissues, Eds. G. P. Chapman et al. pp. 197-209, Longman, N.Y. (pollen); Kaeppler et al. (1990) Plant Cell Reports 9:415-418; Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255; and Christou and Ford (1995) Annals of Botany 75:745-750 (maize via Agrobacterium tumefaciens) all of which are herein incorporated by reference. The cells which have been transformed may be grown into plants in accordance with conventional ways as is discussed in more detail below.

B. Transfection of Prokaryotes, Lower Eukaryotes, and Animal Cells

Animal and lower eukaryotic (e.g., yeast) and prokaryotic host cells are competent or rendered competent for transfection by various means well known in the art. There are several well-known methods of introducing DNA into animal cells. These include: calcium phosphate precipitation, fusion of the recipient cells with bacterial protoplasts containing the DNA, treatment of the recipient cells with liposomes containing the DNA, DEAE dextran, electroporation, biolistics, and micro-injection of the DNA directly into the cells. The transfected cells are cultured by means well known in the art. Kuchler, R. J., Biochemical Methods in Cell Culture and Virology, Dowden, Hutchinson and Ross, Inc. (1977).

Transgenic Plant Regeneration

Plant cells which directly result or are derived from the nucleic acid introduction techniques can be cultured to regenerate a whole plant which possesses the introduced genotype. See, for example, McCormick et al. (1986) Plant Cell Reports 5:81-84. Such regeneration techniques often rely on manipulation of certain phytohormones in a tissue culture growth medium. Plants cells can be regenerated, e.g., from single cells, callus tissue or leaf discs according to standard plant tissue culture techniques. It is well known in the art that various cells, tissues, and organs from almost any plant can be successfully cultured to regenerate an entire plant. Plant regeneration from cultured protoplasts is described in Evans et al. (1983) Protoplasts Isolation and Culture, Handbook of Plant Cell Culture, Macmillan Publishing Company, New York, pp.124-176; and Binding (1985) Regeneration of Plants, Plant Protoplasts, CRC Press, Boca Raton, pp. 21-73.

The regeneration of plants from either single plant protoplasts or various explants is well known in the art. See, for example, Methods for Plant Molecular Biology, A. Weissbach and H. Weissbach, eds., Academic Press, Inc., San Diego, Calif. (1988). This regeneration and growth process includes the steps of selection of transformant cells and shoots, rooting the transformant shoots and growth of the plantlets in soil. For maize cell culture and regeneration see generally, The Maize Handbook, Freeling and Walbot, Eds., Springer, New York (1994); Corn and Corn Improvement, 3^{rd} edition, Sprague and Dudley Eds., American Society of Agronomy, Madison, Wis. (1988). For transformation and regeneration of maize, see Gordon-Kamm et al. (1990) The Plant Cell 2:603-618.

The regeneration of plants from leaf explants containing the polynucleotide of the present invention introduced by Agrobacterium can be achieved as described by Horsch et al. (1985) Science 227:1229-1231. In this procedure, transformants are grown in the presence of a selection agent and in a medium that induces the regeneration of shoots in the plant species being transformed as described by Fraley et al. (1983) PNAS (USA) 80:4803. This procedure typically produces shoots within two to four weeks and these transformant shoots are then transferred to an appropriate root-inducing medium containing the selective agent and an antibiotic to prevent bacterial growth. Transgenic plants of the present invention may be fertile or sterile.

These plants may then be grown, and either pollinated with the same transformed strain or different strains, and the resulting hybrid having the desired phenotypic characteristic identified. Two or more generations may be grown to ensure that the subject phenotypic characteristic is stably maintained and inherited and then seeds harvested to ensure the desired phenotype or other property has been achieved.

One of skill will recognize that after the recombinant expression cassette is stably incorporated in transgenic plants and confirmed to be operable, it can be introduced into other plants by sexual crossing. Any of a number of standard breeding techniques can be used, depending upon the species to be crossed. In vegetatively propagated crops, mature transgenic plants can be propagated by the taking of cuttings or by tissue culture techniques to produce multiple identical plants. Selection of desirable transgenics is made and new varieties are obtained and propagated vegetatively for commercial use. In seed propagated crops, mature transgenic plants can be self-crossed to produce a homozygous inbred plant. The inbred plant produces seed containing the newly introduced heterologous nucleic acid. These seeds can be grown to produce plants that would produce the selected phenotype. Parts obtained from the regenerated plant, such as flowers, seeds, leaves, branches, fruit, and the like are included in the invention, provided that these parts comprise cells comprising the isolated nucleic acid of the present invention. Progeny, variants, and mutants of the regenerated plants are also included within the scope of the invention, provided that these parts comprise the introduced nucleic acid sequences. Transgenic plants expressing a polynucleotide of the present invention can be screened for transmission of the nucleic acid of the present invention by, for example, standard immunoblot and DNA detection techniques. Expression at the RNA level can be determined initially to identify and quantitate expression-positive plants. Standard techniques for RNA analysis can be employed and include PCR amplification assays using oligonucleotide primers designed to amplify only the heterologous RNA templates and solution hybridization assays using heterologous nucleic acid-specific probes. The RNA-positive plants can then analyzed for protein expression by Western immunoblot analysis using the specifically reactive antibodies of the present invention. In addition, in situ hybridization and immunocytochemistry according to standard protocols can be done using heterologous nucleic acid specific polynucleotide probes and antibodies, respectively, to localize sites of expression within transgenic tissue. Generally, a number of transgenic lines are usually screened for the incorporated nucleic acid to identify and select plants with the most appropriate expression profiles.

Transgenic plants of the present invention can be homozygous for the added heterologous nucleic acid; i.e., a transgenic plant that contains two added nucleic acid sequences, one gene at the same locus on each chromosome of a chromosome pair. A homozygous transgenic plant can be obtained by sexually mating (selfing) a heterozygous transgenic plant that contains a single added heterologous nucleic acid, germinating some of the seed produced and analyzing the resulting plants produced for altered expression of a polynucleotide of the present invention relative to a control plant (i.e., native, non-transgenic). Back-crossing to a parental plant and out-crossing with a non-transgenic plant are also contemplated.

Modulating Polypeptide Levels and/or Composition

The present invention further provides a method for modulating (i.e., increasing or decreasing) the concentration or ratio of the polypeptides of the present invention in a plant or part thereof. Modulation can be effected by increasing or decreasing the concentration and/or the ratio of the polypeptides of the present invention in a plant. The method comprises introducing into a plant cell a recombinant expression cassette comprising a polynucleotide of the present invention as described above to obtain a transgenic plant cell, culturing the transgenic plant cell under transgenic plant cell growing conditions, and inducing or repressing expression of a polynucleotide of the present invention in the transgenic plant cell for a time sufficient to modulate concentration and/or the ratios of the polypeptides in the transgenic plant or plant part generated from the transgenic plant cell.

In some embodiments, the concentration and/or ratios of polypeptides of the present invention in a plant may be modulated by altering, in vivo or in vitro, the promoter of a gene to up- or down-regulate gene expression. In some embodiments, the coding regions of native genes of the present invention can be altered via substitution, addition, insertion, or deletion to decrease activity of the encoded enzyme. See, e.g., Kmiec, U.S. Pat. No. 5,565,350; Zarling et al., PCT/US93/03868. And in some embodiments, an isolated nucleic acid (e.g., a vector) comprising a promoter sequence is transfected into a plant cell. Subsequently, a plant cell comprising the promoter operably linked to a polynucleotide of the present invention is selected for by means known to those of skill in the art such as, but not limited to, Southern blot, DNA sequencing, or PCR analysis using primers specific to the promoter and to the gene and detecting amplicons produced therefrom. A plant or plant part altered or modified by the foregoing embodiments is grown under plant forming conditions for a time sufficient to modulate the concentration and/or ratios of polypeptides of the present invention in the plant. Plant forming conditions are well known in the art and discussed briefly, supra.

In general, concentration or the ratios of the polypeptides is increased or decreased by at least 5%, 10%, 20%, 30%, 40%, 50%, 60%, 70%, 80%, or 90% relative to a native control plant, plant part, or cell lacking the aforementioned recombinant expression cassette. Modulation in the present invention may occur during and/or subsequent to growth of the plant to the desired stage of development. Modulating nucleic acid expression temporally and/or in particular tissues can be controlled by employing the appropriate promoter operably linked to a polynucleotide of the present invention in, for example, sense or antisense orientation as discussed in greater detail, supra. Induction of expression of a polynucleotide of the present invention can also be controlled by exogenous administration of an effective amount of inducing compound. Inducible promoters and inducing compounds which activate expression from these promoters are well known in the art. In some embodiments, the polypeptides of the present invention are modulated in monocots, for example, maize.

Targeted Modification of a Polynucleotide of Interest

The Tˆ T translesion synthesis activity of RAD30/Polη, coupled with a modification template comprising at least one Tˆ T dimer, can be used to introduce specific, heritable, targeted modifications to any polynucleotide target sequence of interest. The modification template can be comprised of DNA, or be a DNA-RNA chimera, PNA or other modified nucleotide polymer. These modifications can be used to enhance or suppress the expression of the sequence of interest. The modifications can be the introduction of point or frameshift mutations in a sequence. Either one or two nucleotides can be inserted or converted by each Tˆ T dimer in the modification template. These targeted modifications can be used created in a UTR, in regulatory sequences and/or in a coding sequence. If in a coding sequence, these modifications could be targeted to either exons or introns. Point mutations can be introduced to convert a codon to a more preferred codon, convert a codon to substitute a different amino acid, convert a codon to introduce a premature stop codon, alter an intron-exon splicing site or any other post-transcriptional processing site, or to alter other regulatory regions such as a promotor or any other UTR. Frameshift mutations can also be generated by the insertion of 1-2 adenines for every Tˆ T dimer in the modification template. More than one site in a target could be modified by designing modification templates comprising more than one Tˆ T dimer, or by using more than one template. The modification template can range anywhere between about 15 nucleotides in length to the full-length of the target polynucleotide of interest, typically the template will be between 15-200 nucleotides in length. The modification template is directed to the target polynucleotide by the shared homology between the sequences, typically the sequences will be identical except where a Tˆ T dimer is incorporated. RAD30/Polη can be introduced prior to, or simultaneously with, the modification template, using standard techniques known in the art. The invention foresees using methods which transiently introduce all necessary components to effect a targeted modification which will result in the production of a non-transgenic host which has stably incorporated a specific, heritable, targeted sequence modification. The invention also foresees the production of transgenic cells, plants, and seeds, which comprise a specific, heritable, targeted modification to a polynucleotide sequence of interest, and may further comprise a RAD30/Polη polynucleotide of the present invention.

Molecular Markers

Genotyping provides a means of distinguishing homologs of a chromosome pair and can be used to differentiate segregants in a plant population. Molecular marker methods can be used for phylogenetic studies, characterizing genetic relationships among crop varieties, identifying crosses or somatic hybrids, localizing chromosomal segments affecting monogenic traits, map based cloning, and the study of quantitative inheritance. The polynucleotide of the present invention may be used to develop molecular markers for various plant populations. See, e.g., Clark, Ed., Plant Molecular Biology: A Laboratory Manual. Berlin, Springer-Verlag, Chapter 7 (1997). For molecular marker methods, see generally, “The DNA Revolution” in: Paterson, A. H., Genome Mapping in Plants (Austin, Tex., Academic Press/R. G. Landis Company, pp.7-21 (1996).

The particular method of genotyping in the present invention may employ any number of molecular marker analytic techniques such as, but not limited to, restriction fragment length polymorphisms (RFLPs). RFLPs are the product of allelic differences between DNA restriction fragments resulting from nucleotide sequence variability. As is well known to those of skill in the art, RFLPs are typically detected by extraction of genomic DNA and digestion with a restriction enzyme. Generally, the resulting fragments are separated according to size and hybridized with a probe; typically a single copy probe. Restriction fragments from homologous chromosomes are revealed, and differences in fragment size among alleles represent an RFLP. Thus, the present invention further provides a means to follow segregation of a gene or nucleic acid of the present invention as well as chromosomal sequences genetically linked to these genes or nucleic acids using such techniques as RFLP analysis. Linked chromosomal sequences are within 50 centiMorgans (cM), often within 40 or 30 cM, or within 20 or 10 cM, or even within 5, 3, 2, or 1 cM of a gene of the present invention.

In the present invention, the nucleic acid probes employed for molecular marker mapping of plant nuclear genomes selectively hybridize, under selective hybridization conditions, to a gene encoding a polynucleotide of the present invention. In some embodiments, the probes are selected from polynucleotides of the present invention. Typically, these probes are cDNA probes or restriction-enzyme treated (e.g., PstI) genomic clones. The length of the probes is discussed in greater detail, supra, but are typically at least 15 bases in length, or at least 20, 25, 30, 35, 40, or 50 bases in length. Generally, however, the probes are less than about 1 kilobase in length. Typically, the probes are single copy probes that hybridize to a unique locus in a haploid chromosome complement. Some exemplary restriction enzymes employed in RFLP mapping are EcoRI, EcoRV, and SstI. As used herein the term “restriction enzyme” includes reference to a composition that recognizes and, alone or in conjunction with another composition, cleaves at a specific nucleotide sequence.

The method of detecting an RFLP comprises the steps of (a) digesting genomic DNA of a plant with a restriction enzyme; (b) electrophoretically separating the digestion product fragments on a gel matrix; (c) hybridizing a labeled nucleic acid probe, under selective hybridization conditions, to said digested genomic DNA; (d) detecting therefrom an RFLP. Other methods of differentiating polymorphic (allelic) variants of polynucleotides of the present invention can be had by utilizing molecular marker techniques well known to those of skill in the art including such techniques as: 1) single stranded conformation analysis (SSCA); 2) denaturing gradient gel electrophoresis (DGGE); 3) RNase protection assays; 4) allele-specific oligonucleotides (ASOs); 5) the use of proteins which recognize nucleotide mismatches, such as the E. coli mutS protein; and 6) allele-specific PCR. Other approaches based on the detection of mismatches between the two complementary DNA strands include clamped denaturing gel electrophoresis (CDGE); heteroduplex analysis (HA); and chemical mismatch cleavage (CMC). Thus, the present invention further provides a method of genotyping comprising the steps of contacting, under stringent hybridization conditions, a sample suspected of comprising a polynucleotide of the present invention with a nucleic acid probe. Generally, the sample is a plant sample; likely, a sample suspected of comprising a polynucleotide of the present invention (e.g., gene, mRNA). The nucleic acid probe selectively hybridizes, under stringent conditions, to a subsequence of a polynucleotide of the present invention comprising a polymorphic marker. Selective hybridization of the nucleic acid probe to the polymorphic marker nucleic acid sequence yields a hybridization complex. Detection of the hybridization complex indicates the presence of that polymorphic marker in the sample. In some embodiments, the nucleic acid probe comprises a polynucleotide of the present invention.

UTRs and Codon Preference

In general, translational efficiency has been found to be regulated by specific sequence elements in the 5′ non-coding or untranslated region (5′ UTR) of the RNA. Positive sequence motifs include translational initiation consensus sequences (Kozak (1987) Nucl. Acids Res. 15:8125) and the 7-methylguanosine cap structure (Drummond et al. (1985) Nucl. Acids Res. 13:7375). Negative elements include stable intramolecular 5′ UTR stem-loop structures (Muesing et al. (1987) Cell 48:691) and AUG sequences or short open reading frames preceded by an appropriate AUG in the 5′ UTR (Kozak, supra; Rao et al. (1988) Mol. Cell. Biol. 8:284). Accordingly, the present invention provides 5′ and/or 3′ untranslated regions for modulation of translation of heterologous coding sequences.

Further, the polypeptide-encoding segments of the polynucleotides of the present invention can be modified to alter codon usage. Altered codon usage can be employed to alter translational efficiency and/or to optimize the coding sequence for expression in a desired host, such as to optimize the codon usage in a heterologous sequence for expression in maize. Codon usage in the coding regions of the polynucleotides of the present invention can be analyzed statistically using commercially available software packages such as “Codon Preference” available from the University of Wisconsin Genetics Computer Group (see Devereaux et al. (1984) Nucl. Acids Res. 12: 387-395) or MacVector 4.1 (Eastman Kodak Co., New Haven, Conn.). Thus, the present invention provides a codon usage frequency characteristic of the coding region of at least one of the polynucleotides of the present invention. The number of polynucleotides that can be used to determine a codon usage frequency can be any integer from 1 to the number of polynucleotides of the present invention as provided herein. Optionally, the polynucleotides will be full-length sequences. An exemplary number of sequences for statistical analysis can be at least 1, 5, 10, 20, 50, or 100.

Sequence Shuffling

The polynucleotides of the present invention can be used in sequence shuffling to generate variants with a desired characteristic, such as altered levels of catalytic activity or altered binding affinity or specificity. Sequence shuffling is described in PCT publication No. WO 97/20078. See also, Zhang, J-H et al. (1997) PNAS (USA) 94:45044509. Generally, sequence shuffling provides a means for generating libraries of polynucleotides having a desired characteristic which can be selected or screened for. Libraries of recombinant polynucleotides are generated from a population of related sequence polynucleotides which comprise sequence regions which have substantial sequence identity and can be homologously recombined in vitro or in vivo. The population of sequence-recombined polynucleotides comprises a subpopulation of polynucleotides which possess desired or advantageous characteristics and which can be selected by a suitable selection or screening method. The characteristics can be any property or attribute capable of being selected for or detected in a screening system, and may include properties of: an encoded protein, a transcriptional element, a sequence controlling transcription, RNA processing, RNA stability, chromatin conformation, translation, or other expression property of a gene or transgene, a replicative element, a protein-binding element, or the like, such as any feature which confers a selectable or detectable property. In some embodiments, the selected characteristic will be a decreased Kₘ and/or increased K_{cat} over the wild-type protein as provided herein. In other embodiments, a protein or polynucleotide generated from sequence shuffling will have a ligand binding affinity greater than the non-shuffled wild-type polynucleotide. The increase in such properties can be at least 110%, 120%, 130%, 140% or at least 150% of the wild-type value.

Generic and Consensus Sequences

Polynucleotides and polypeptides of the present invention further include those having: (a) a generic sequence of at least two homologous polynucleotides or polypeptides, respectively, of the present invention; and, (b) a consensus sequence of at least three homologous polynucleotides or polypeptides, respectively, of the present invention. The generic sequence of the present invention comprises each species of polypeptide or polynucleotide embraced by the generic polypeptide or polynucleotide sequence, respectively. The individual species encompassed by a polynucleotide having an amino acid or nucleic acid consensus sequence can be used to generate antibodies or produce nucleic acid probes or primers to screen for homologs in other species, genera, families, orders, classes, phyla, or kingdoms. For example, a polynucleotide having a consensus sequence from a gene family of Zea mays can be used to generate antibody or nucleic acid probes or primers to other Gramineae species such as wheat, rice, or sorghum. Alternatively, a polynucleotide having a consensus sequence generated from orthologous genes can be used to identify or isolate orthologs of other taxa. Typically, a polynucleotide having a consensus sequence will be at least 9, 10, 15, 20, 25, 30, or 40 amino acids in length, or 20, 30, 40, 50, 100, or 150 nucleotides in length. As those of skill in the art are aware, a conservative amino acid substitution can be used for amino acids which differ amongst aligned sequence but are from the same conservative substitution group as discussed above. Optionally, no more than 1 or 2 conservative amino acids are substituted for each 10 amino acid length of consensus sequence.

Similar sequences used for generation of a consensus or generic sequence include any number and combination of allelic variants of the same gene, orthologous, or paralogous sequences as provided herein. Optionally, similar sequences used in generating a consensus or generic sequence are identified using the BLAST algorithm's smallest sum probability (P(N)). Various suppliers of sequence-analysis software are listed in Ch. 7 of Current Protocols in Molecular Biology, F. M. Ausubel et al., Eds., Current Protocols, a joint venture between Greene Publishing Associates, Inc. and John Wiley & Sons, Inc. (Supplement 30). A polynucleotide sequence is considered similar to a reference sequence if the smallest sum probability in a comparison of the test nucleic acid to the reference nucleic acid is less than about 0.1, typically less than about 0.01, or 0.001, and optionally less than about 0.0001, or 0.00001. Similar polynucleotides can be aligned and a consensus or generic sequence generated using multiple sequence alignment software available from a number of commercial suppliers such as the Genetics Computer Group (Madison, Wis.) PILEUP software, Vector NTI (North Bethesda, Md.) ALIGNX, or Genecode (Ann Arbor, Mich.) SEQUENCHER. Conveniently, default parameters of such software can be used to generate consensus or generic sequences.

Assays for Compounds that Modulate Enzymatic Activity or Expression

The present invention also provides means for identifying compounds that bind to (e.g., substrates), and/or increase or decrease (i.e., modulate) the enzymatic activity of, catalytically active polypeptides of the present invention. The method comprises contacting a polypeptide of the present invention with a compound whose ability to bind to or modulate enzyme activity is to be determined. The polypeptide employed will have at least 20%, 30%, 40%, or at least 50% or 60%, or at least 70% or 80% of the specific activity of the native, full-length polypeptide of the present invention (e.g., enzyme). Generally, the polypeptide will be present in a range sufficient to determine the effect of the compound, typically about 1 nM to 10 μM. Likewise, the compound will be present in a concentration of from about 1 nM to 10 μM. Those of skill will understand that such factors as enzyme concentration, ligand concentrations (i.e., substrates, products, inhibitors, activators), pH, ionic strength, and temperature will be controlled so as to obtain useful kinetic data and determine the presence of absence of a compound that binds or modulates polypeptide activity. Methods of measuring enzyme kinetics is well known in the art. See, e.g., Segel, Biochemical Calculations, 2^{nd} ed., John Wiley and Sons, New York (1976).

Isolation of DNA Repair Factors

The present invention also provides means for identifying other factors involved in DNA repair. Many methods for identifying and characterizing protein-protein interactions are known in the art. For example, the polynucleotide of the present invention can be used as “bait” in a yeast two-hybrid screen against a cDNA library to identify interacting factors. The assay is based on the functional reconstitution of a transcriptional activator. Methods for constructing a tagged cDNA library and bait constructs are well known in the art. See, e.g. Ch. 20.1 Current Protocols in Molecular Biology, F. M. Ausubel et al., Eds., Current Protocols, Greene Publishing Associates, Inc. and John Wiley & Sons, Inc. Screening components are also commercially available, for example the MATCHMAKER Two-hybrid System Protocol from CLONETECH. Once interacting factors are identified, functional domains and the binding interface can be further characterized with the yeast two-hybrid system by testing the ability of fragments or mutated sequences to reconstitute the transcriptional activator.

The Ras recruitment system (RRS) is another two-hybrid system that can be used to identify and characterize protein-protein interactions. This system is based on the fact that Ras must be localized to the plasma membrane in order to function. This screen is based on Ras membrane localization and activation achieved through the interaction of two hybrid proteins as described in Broder et al. (1998) Current Biology 8(20):1121-1124.

Factors that interact with the polypeptide of the present invention can also be isolated using a co-immunoprecipitation assay. Under non-denaturing conditions, a lysate is made of cells expressing the polypeptide of the present invention. An antibody directed against the polypeptide of the present invention is used in an immunoprecipitation assay in non-denaturing conditions. Under the proper conditions, the polypeptide of the present invention and any factors bound to it are co-immunoprecipitated and further analyzed by SDS polyacrylamide gel electrophoresis (PAGE) and other protein characterization methods known in the art. See, for example, Harlow and Lane, Antibodies, Cold Spring Harbor Press; and Ch. 10.16 Current Protocols in Molecular Biology, F. M. Ausubel et al. (supra).

Another method is to utilize a fusion tag for affinity purification, for example the polynucleotide of the present invention can be put in a GST-fusion construct and GST-fusion protein expressed. This technique is also known as GST pulldown purification. The GST fusion protein is first purified on glutathione-agarose beads. The bead-bound fusion protein is used as “bait” in order to affinity purify factors that bind to the protein. See, e.g. Ch. 20.2 Current Protocols in Molecular Biology, F. M. Ausubel et al., Eds. (supra).

Detection of Nucleic Acids

The present invention further provides methods for detecting a polynucleotide of the present invention in a nucleic acid sample suspected of containing a polynucleotide of the present invention, such as a plant cell lysate, for example, a lysate of maize. In some embodiments, a cognate gene of a polynucleotide of the present invention or portion thereof can be amplified prior to the step of contacting the nucleic acid sample with a polynucleotide of the present invention. The nucleic acid sample is contacted with the polynucleotide to form a hybridization complex. The polynucleotide hybridizes under stringent conditions to a gene encoding a polypeptide of the present invention. Formation of the hybridization complex is used to detect a gene encoding a polypeptide of the present invention in the nucleic acid sample. Those of skill will appreciate that an isolated nucleic acid comprising a polynucleotide of the present invention should lack cross-hybridizing sequences in common with non-target genes that would yield a false positive result. Detection of the hybridization complex can be achieved using any number of well known methods. For example, the nucleic acid sample, or a portion thereof, may be assayed by hybridization formats including but not limited to, solution phase, solid phase, mixed phase, or in situ hybridization assays.

Detectable labels suitable for use in the present invention include any composition detectable by spectroscopic, radioisotopic, photochemical, biochemical, immunochemical, electrical, optical or chemical means. Useful labels in the present invention include biotin for staining with labeled streptavidin conjugate, magnetic beads, fluorescent dyes, radiolabels, enzymes, and calorimetric labels. Other labels include ligands which bind to antibodies labeled with fluorophores, chemiluminescent agents, and enzymes. Labeling the nucleic acids of the present invention is readily achieved such as by the use of labeled PCR primers.

Although the present invention has been described in some detail by way of illustration and example for purposes of clarity of understanding, it will be obvious that certain changes and modifications may be practiced within the scope of the appended claims.

### EXAMPLE 1

This example describes the construction of a cDNA library.

The RNA for SEQ ID NO: 1 was isolated from maize night harvested ear shoot with husk at the V-12 stage. Total RNA can be isolated from maize tissues with TRIZOL Reagent (Life Technologies, Inc. Gaithersburg, Md.) using a modification of the guanidine isothiocyanate/acid-phenol procedure described by Chomczynski and Sacchi (1987) Anal. Biochem. 162:156. In brief, plant tissue samples are pulverized in liquid nitrogen before the addition of the TRIZOL Reagent, and then further homogenized with a mortar and pestle. Addition of chloroform followed by centrifugation is conducted for separation of an aqueous phase and an organic phase. The total RNA is recovered by precipitation with isopropyl alcohol from the aqueous phase.

The selection of poly(A)+RNA from total RNA can be performed using POLYATTRACT system (Promega Corp., Madison, Wis.). Biotinylated oligo(dT) primers are used to hybridize to the 3′ poly(A) tails on mRNA. The hybrids are captured using streptavidin coupled to paramagnetic particles and a magnetic separation stand. The mRNA is then washed at high stringency conditions and eluted by RNase-free deionized water.

cDNA synthesis and construction of unidirectional cDNA libraries can be accomplished using the SUPERSCRIPT Plasmid System (Life Technologies, Inc. Gaithersburg, Md.). The first strand of cDNA is synthesized by priming with an oligo(dT) primer containing a NotI site. The reaction is catalyzed by SUPERSCRIPT Reverse Transcriptase II at 45° C. The second strand of cDNA is labeled with alpha-³²P-dCTP and a portion of the reaction analyzed by agarose gel electrophoresis to determine cDNA sizes. cDNA molecules smaller than 500 base pairs and unligated adapters are removed by SEPHACRYL-S400 (Pharmacia) chromatography. The selected cDNA molecules are ligated into pSPORT1 vector (Life Technologies, Inc. Gaithersburg, Md.) in between NotI and Sa/I sites.

Alternatively, cDNA libraries can be prepared by any one of many methods available. For example, the cDNAs may be introduced into plasmid vectors by first preparing the cDNA libraries in Uni-ZAP™ XR vectors according to the manufacturer's protocol (Stratagene Cloning Systems, La Jolla, Calif.). The Uni-ZAP™ XR libraries are converted into plasmid libraries according to the protocol provided by Stratagene. Upon conversion, cDNA inserts will be contained in the pBluescript plasmid vector. In addition, the cDNAs may be introduced directly into precut Bluescript II SK(+) vectors (Stratagene) using T4 DNA ligase (New England Biolabs), followed by transfection into DH10B cells according to the manufacturer's protocol (GIBCO BRL Products). Once the cDNA inserts are in plasmid vectors, plasmid DNAs are prepared from randomly picked bacterial colonies containing recombinant pBluescript plasmids, or the insert cDNA sequences are amplified via polymerase chain reaction using primers specific for vector sequences flanking the inserted cDNA sequences. Amplified insert DNAs or plasmid DNAs are sequenced in dye-primer sequencing reactions to generate partial cDNA sequences (expressed sequence tags or “ESTs”; see Adams et al. (1991) Science 252:1651-1656). The resulting ESTs are analyzed using a Perkin Elmer Model 377 fluorescent sequencer.

### EXAMPLE 2

This example describes cDNA sequencing and library subtraction. Individual colonies can be picked and DNA prepared either by PCR with M13 forward primers and M13 reverse primers, or by plasmid isolation. cDNA clones can be sequenced using M13 reverse primers.

cDNA libraries are plated out on 22×22 cm² agar plate at density of about 3,000 colonies per plate. The plates are incubated in a 37° C. incubator for 12-24 hours. Colonies are picked into 384-well plates by a robot colony picker, Q-bot (GENETIX Limited). These plates are incubated overnight at 37° C. Once sufficient colonies are picked, they are pinned onto 22×22 cm² nylon membranes using Q-bot. Each membrane holds 9,216 or 36,864 colonies. These membranes are placed onto an agar plate with an appropriate antibiotic. The plates are incubated at 37° C. overnight.

After colonies are recovered on the second day, these filters are placed on filter paper prewetted with denaturing solution for four minutes, then incubated on top of a boiling water bath for an additional four minutes. The filters are then placed on filter paper prewetted with neutralizing solution for four minutes. After excess solution is removed by placing the filters on dry filter papers for one minute, the colony side of the filters is placed into Proteinase K solution and incubated at 37° C. for 40-50 minutes. The filters are placed on dry filter papers to dry overnight. DNA is then cross-linked to nylon membrane by UV light treatment.

Colony hybridization is conducted as described by Sambrook, J. et al. (in Molecular Cloning: A Laboratory Manual, 2^{nd} Edition). The following probes can be used in colony hybridization:

1. First strand cDNA from the same tissue as the source library to remove the most redundant clones.

2. 48-192 most redundant cDNA clones from the same library based on previous sequencing data.

3. 192 most redundant cDNA clones in the entire maize sequence database.

4. A SaI-A20 oligo nucleotide: TCG ACC CAC GCG TCC GAA AAA AAA AAA AAA AAA AAA, listed in SEQ ID NO. 3, removes clones containing a poly A tail but no cDNA.

5. cDNA clones derived from rRNA.

The image of the autoradiograph is scanned into computer and the signal intensity and cold colony addresses of each colony is analyzed. Re-arraying of cold-colonies from 384 well plates to 96 well plates is conducted using the Q-bot robot.

### EXAMPLE 3

This example describes the mapping of the maize RAD30/Polη polynucleotide sequence exemplified in SEQ ID NO: 1.

A maize EST clone (clone ID # CMTMX27) was found in a cDNA library prepared from mRNA isolated from maize night harvested ear shoot with husk, V-12 stage. This clone had an open reading frame of about 2.5 kb that showed a deduced protein sequence of about 649 amino acids having homology to known RAD30/Poη sequences. This clone has been mapped to maize chromosome 3 as described below.

Probe fragments are generated that are identical to the original maize RAD30/Polη genes. In order to make these probe fragments, hybridization oligonucleotide primers specific to unique portions of the RAD30/Polη genes are synthesized and used in conjunction with an M13 universal sequencing primer to PCR amplify probe fragments from the RAD30/Polη gene sequence. These fragments, which extend from just downstream of the translation stop codon to the end of the poly(A) tail of the cDNA sequences, are used as probes against two maize populations and map positions are determined.

Southern hybridizations are carried out using two different maize populations generated as part of a breeding program. Population 1 (MARSA—Marker Assisted Recombination Selection population), an F4, is generated from crosses of the lines R03×N46, and contains 200 individuals as part of the mapping family. Population 2 (ALEB9), an F2, is generated from crosses of the lines R67×P38 and contains 240 individuals. DNA is isolated from each individual by a CTAB extraction method (Saghai-Maroof et al. (1994) PNAS (USA) 81:8014-8018) and then digested individually with restriction enzymes BamHI, HindIII, EcoRI and EcoRV. Digests are separated on agarose gels and transferred to membranes (Southern (1975) J. Mol. Biol. 98:503-517) prior to hybridization (Helentjaris et al. (1985) Plant Mol. Biol. 5:109-118) with an array of probes to establish the basic RFLP map. Population 1 membranes are hybridized using 179 RFLP probes, while population 2 membranes are hybridized using 115 RFLP probes. After hybridization the membranes are exposed to x-ray film for an appropriate length of time to be visually scored. All data is entered into an electronic database and map positions of the RFLP probes (Evola et al. (1986) Theor. Appl. Genet. 71:765-771) are determined using MAPMAKER (Lincoln et al. (1993) in Constructing Genetic Linkage Maps with MAPMARKER/EXP Version 3.0: A Tutorial and Reference Manual, Whitehead Institute for Biomedical Research, Cambridge, Mass.) and a map is constructed for each population.

### EXAMPLE 4

This example describes identification of the gene from a computer homology search.

Gene identities can be determined by conducting BLAST (Altschul, S F et al., (1990) J. Mol. Biol. 215:403-410) searches under default parameters for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The cDNA sequences are analyzed for similarity to all publicly available DNA sequences contained in the “nr” database using the BLASTN algorithm. The DNA sequences are translated in all reading frames and compared for similarity to all publicly available protein sequences contained in the “nr” database using the BLASTX algorithm (Gish and States (1993) Nature Genetics 3:266-272) provided by the NCBI. In some cases, the sequencing data from two or more clones containing overlapping segments of DNA are used to construct contiguous DNA sequences.

Sequence alignments and percent identity calculations can be performed using the GAP algorithm in Version 10 of GCG, or the Megalign program of the LASERGENE bioinformatics computing suite (DNASTAR Inc., Madison, Wis.). Multiple alignment of the sequences can be performed using the PileUp program in GCG, or the Clustal method of alignment (Higgins and Sharp (1989) CABIOS 5:151-153) with the default parameters (GAP PENALTY=10, GAP LENGTH PENALTY=10). Default parameters for pairwise alignments using the Clustal method are KTUPLE 1, GAP PENALTY=3, WINDOW=5 and DIAGONALS SAVED=5.

### EXAMPLE 5

This example provides methods of plant transformation and regeneration using the polynucleotides of the present invention, as well as a method to determine their effect on transformation efficiency.

A. Transformation by Particle Bombardment

Transformation of Maize Embryos

Transformation of a RAD30/Polη construct along with a marker expression cassette (for example, UBI::moPAT-GFPm::pinII) into genotype Hi-II follows a well-established bombardment transformation protocol used for introducing DNA into the scutellum of immature maize embryos (Songstad et al. (1996) In Vitro Cell Dev. Biol. Plant 32:179-183). It is noted that any suitable method of transformation can be used, such as Agrobacterium-mediated transformation and many other methods. To prepare suitable target tissue for transformation, ears are surface sterilized in 50% Chlorox bleach plus 0.5% Micro detergent for 20 minutes, and rinsed two times with sterile water. The immature embryos (approximately 1-1.5 mm in length) are excised and placed embryo axis side down (scutellum side up), 25 embryos per plate. These are cultured onto medium containing N6 salts, Erikkson's vitamins, 0.69 g/L proline, 2 mg/L 2,4-D and 3% sucrose. After 4-5 days of incubation in the dark at 28° C., embryos are removed from the first medium and cultured onto similar medium containing 12% sucrose. Embryos are allowed to acclimate to this medium for 3 h prior to transformation. The scutellar surface of the immature embryos is targeted using particle bombardment. Embryos are transformed using the PDS-1000 Helium Gun from Bio-Rad at one shot per sample using 650PSI rupture disks. DNA delivered per shot averages approximately 0.1667 μg. Following bombardment, all embryos are maintained on standard maize culture medium (N6 salts, Erikkson's vitamins, 0.69 g/L proline, 2 mg/L 2,4-D, 3% sucrose) for 2-3 days and then transferred to N6-based medium containing 3 mg/L Bialaphos®. Plates are maintained at 28° C. in the dark and are observed for colony recovery with transfers to fresh medium every two to three weeks. After approximately 10 weeks of selection, selection-resistant GFP positive callus clones can be sampled for presence of RAD30/Polη mRNA and/or protein. Positive lines are transferred to 288J medium, an MS-based medium with lower sucrose and hormone levels, to initiate plant regeneration. Following somatic embryo maturation (2-4 weeks), well-developed somatic embryos are transferred to medium for germination and transferred to the lighted culture room. Approximately 7-10 days later, developing plantlets are transferred to medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to Classic™ 600 pots (1.6 gallon) and grown to maturity. Plants are monitored for expression of RAD30/Polη mRNA and/or protein. Recovered colonies and plants can be scored based on GFP visual expression, leaf painting sensitivity to a 1% application of Ignite® herbicide, and molecular characterization via PCR and Southern analysis.

Transformation of Soybean Embryos

Soybean embryos are bombarded with a plasmid containing a nucleotide sequence encoding a protein of the present invention operably linked to a selected promoter as follows. To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can be maintained in 35 ml liquid media on a rotary shaker, 150 rpm, at 26° C. with fluorescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein et al. (1987) Nature 327:70-73; and U.S. Pat. No. 4,945,050). A DuPont Biolistic PDS1000/HE instrument (helium retrofit) can be used for these transformations.

A selectable marker gene that can be used to facilitate soybean transformation is a transgene composed of the 35S promoter from Cauliflower Mosaic Virus (Odell et al. (1985) Nature 313:810-812), the hygromycin phosphotransferase gene from plasmid pJR225 (from E. coli; Gritz et al. (1983) Gene 25:179-188), and the 3′ region of the nopaline synthase gene from the T-DNA of the Ti plasmid of Agrobacterium tumefaciens. The expression cassette comprising the nucleotide sequence encoding a protein of the present invention operably linked to the selected promoter can be isolated as a restriction fragment. This fragment can then be inserted into a unique restriction site of the vector carrying the marker gene.

DNA is prepared for introduction into the plant cells as follows. To 50 μl of a 60 mg/ml 1 μm gold particle suspension is added (in order): 5 μl DNA (1 μg/μl), 20 μl spermidine (0.1 M), and 50 μl CaCl2 (2.5 M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos. Selectable marker resistant putative events can be screened for the presence or expression of the transgene by standard nucleic acid or protein techniques.

B. Transformation by Agrobacterium

Transformation of a RAD30/Polη cassette along with UBI::moPAT˜moGFP::pinII into a maize genotype such as Hi-II (or inbreds such as Pioneer Hi-Bred International, Inc. proprietary inbreds N46 and P38) is also done using the Agrobacterium mediated DNA delivery method, as described by U.S. Pat. No. 5,981,840 with the following modifications. Again, it is noted that any suitable method of transformation can be used, such as particle-mediated transformation, as well as many other methods. Agrobacterium cultures are grown to log phase in liquid minimal-A medium containing 100 μM spectinomycin. Embryos are immersed in a log phase suspension of Agrobacteria adjusted to obtain an effective concentration of 5×10⁸ cfu/ml. Embryos are infected for 5 minutes and then co-cultured on culture medium containing acetosyringone for 7 days at 20° C. in the dark. After 7 days, the embryos are transferred to standard culture medium (MS salts with N6 macronutrients, 1 mg/L 2,4-D, 1 mg/L Dicamba, 20 g/L sucrose, 0.6 g/L glucose, 1 mg/L silver nitrate, and 100 mg/L carbenicillin) with 3 mg/L Bialaphos® as the selective agent. Plates are maintained at 28° C. in the dark and are observed for colony recovery with transfers to fresh medium every two to three weeks. Positive lines are transferred to an MS-based medium with lower sucrose and hormone levels, to initiate plant regeneration. Following somatic embryo maturation (2-4 weeks), well-developed somatic embryos are transferred to medium for germination and transferred to the lighted culture room. Approximately 7-10 days later, developed plantlets are transferred to medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to Classic™ 600 pots (1.6 gallon) and grown to maturity. Recovered colonies and plants can be scored based on GFP visual expression, leaf painting sensitivity to a 1% application of Ignite® herbicide, and molecular characterization via PCR and Southern analysis.

C. Determining Changes in Transformation Efficiency

Transformation frequency may be improved by introducing RAD30/Polη using Agrobacterium or particle bombardment. Plasmids described in this example are used to transform immature embryos using particle delivery or the Agrobacterium. The effect of RAD30/Polη can be measured by comparing the transformation efficiency of RAD30/Polη constructs co-transformed with GFP constructs to the transformation efficiency of control GFP constructs only. Source embryos from individual ears will be split between the two test groups in order to minimize any effect on transformation efficiency due differences in starting material. Selectable marker resistant GFP+colonies are counted using a GFP microscope and transformation frequencies are determined (percentage of initial target embryos from which at least one GFP-expressing, selectable marker-resistant multicellular transformed event grows). In both particle gun experiments and Agrobacterium experiments, transformation frequencies may be increased in the RAD30/Polη treatment group.

D. Transient Expression of the RAD30/Polη Polynucleotide Product

It may be desirable to transiently express RAD30/Polη in order to introduce a targeted sequence modification of another polynucleotide of interest without incorporating the RAD30/Polη polynucleotide into the genome of the target cell. This can be done by delivering RAD30/Polη 5′capped polyadenylated RNA or expression cassettes containing RAD30/Polη DNA. These molecules can be delivered using a biolistics particle gun. For example, 5′ capped polyadenylated RAD30/Polη mRNA can easily be made in vitro using Ambion's mMessage mMachine kit. Following the procedure outlined above, RAD30/Polη RNA or DNA is co-delivered along with a modification template, comprising at least one Tˆ T dimer, directed to a polynucleotide of interest. The cells receiving the RNA or expression cassette will transiently express RAD30/Polη which will facilitate the modification of the target polynucleotide of interest. Plants regenerated from these embryos can then be screened for the presence of the modification of interest. Alternatively, RAD30/Polη polypeptide can be directly introduced into the target cell by any means known in the art, such as microinjection, lipofusion, and the like.

### EXAMPLE 6

This example indicates structural or functional domains found in the RAD30/REV1/DinB/UmuC/DNA Polymerase q (eta)/DNA damage inducible protein gene family members. The amino acid sequence (SEQ ID NO: 2) encoded by the Sa/I/NotI fragment of the maize RAD30/Polη clone CMTMX27 (SEQ ID NO: 1) obtained from night harvested ear shoot with husk at the V-12 stage is shown. The RAD30/Polη polypeptide of the present invention contains five domains, motifs I-V, conserved from bacteria to humans as illustrated in this example (See also McDonald, J P et al. (1999) Genomics 60:20-30). These conserved motifs are clustered in the amino-terminal region of the protein, as is the case with other RAD30-like proteins. Motif I which extends from R12 through R30, and Motif II extending from G51 through I80, have not yet had functions assigned to them, but are presumably critical for some aspect of RAD30/Polη function as they are conserved in prokaryotes, archaea, and eukaryotes. Motif III, amino acids E115 through L126, comprises a SIDEXX box domain, conserved in all known Polη, involved in binding Mg++, and which may serve as the catalytic site of the enzyme. Motif IV, amino acids C206 through V232, and Motif V, amino acids V246 through L260, each contain a helix-hairpin-helix domain found in other Rad30-like proteins and which are associated with DNA binding. The sequence also contains two putative nuclear localization signal sequences at positions K354-K369 and A511-K525 in the amino acid sequence.

### Amino Acid Sequence and Conserved Domains

1 MPVARPEPQE PRVIAHVDMD CFYVQVEQRR NPALRGQPTA VVQYNGWKGG I

51 GLIAVSYEAR GFGVKRSMRG DEAKRVCPGI NLVQVPVARG KADLNLYRSA II

101 GAEVVAILAS KGKCERASID EVYLDLTDAA KEMLLQAPPD SPEGIFMEAA III

151 KSNILGLPAD ASEKEKNVRA WLCQSEADYQ DKLLACGAII VAQLRVRVLE

201 ETQFTCSAGI AHNKMLAKLV SGMHKPAQQT VVPSSSVQDL LASLPVKKMK IV

251 QLGGKLGSSL QDDLGVETIG DLLSFTEEKL QEQYGVNTGT WLWKTARGIS V

301 GEEVEDRLLP KSHGCGKTFP GPRALKYSAS VKGWLDQLCE ELSERIQSDL

351 NQNKRI,AQTL 7 TLHARAFKKN EHDSMKKFPS KSCPLRYGTG KIQEDAMRLF NLS

401 ESGLHEFLES QNTGWGITSL SVTASKIFDI PSGTSSILRY IKGPSSAAAL

451 TIPDSPSSAA ALAIPDSSFV PEDPSLDNDV FVEPIHEEEC QPSTSEKEDD

501 NNTHSASAFS Av:KKCRANE-K.RISKLPGVQ GTSSILKFLS RGQSTLHEKR NLS

551 KSDGLICSHQ GPGSSSEAYK AGAHNVPAEA EDRNNTNSCA EPSGSNTWTF

601 NLQDIDPAVV EELPPEIQRE IQGWVRPSKH PITKRRGSTI SSYFPPARS-

Amino acid sequence (SEQ ID NO: 2) deduced from the nucleotide sequence of full-length cDNA clone CMTMX27 (SEQ ID NO: 1). The conserved motifs are shown in differently formatted text as further described. Conserved motif I is shown in italicized bold text. Motif II is shown in bold text. Motif III is shown in underlined bold text and contains the catalytic site residues SIDE conserved in the Rad30/Polη family. Motifs IV and V are shown in italics and underlined italics respectively. Motifs IV and V contain the Helix-hairpin-Helix (HhH) DNA binding domains found in many DNA repair proteins. Two putative nuclear localization signal (NLS) sequences are also highlighted.

### EXAMPLE 7

This example describes the synthesis and construction of polynucleotides comprising at least one Tˆ T cyclobutane dimer. These polynucleotides can be used as primers to direct the targeted modification of a polynucleotide sequence of interest or as a substrate in assays for RAD30/Polη activity.

Polynucleotides comprising at least one Tˆ T cyclobutane dimer are constructed and purified using the published protocols of Murata et al. (1990) Nucl. Acids Res. 18:7279-7286; and Smith and Taylor (1993) J. Biol. Chem. 268:11143-11151.

Briefly, a Tˆ T dimer can be created in a polynucleotide by either UV irradiation of a polynucleotide containing two adjacent thymidines, or by synthesis of oligonucleotides using a dinucleotide thymidine dimer building block. One can synthesize several oligonucleotides, with at least one containing a thymidine dimer, and ligate the oligonucleotides to form longer polynucleotide sequences.

The dinucleotide building block is constructed via chemical reactions between modified thymidines, followed by chromatographic purifications. Once the thymidines are chemically linked, photodimerization is performed using acetone as a sensitizer. The desired thymine dimer cyclobutane isomer is purified and used for DNA synthesis on a standard DNA synthesizer.

### EXAMPLE 8

This example describes a method for detecting RAD30/Polη activity.

RAD30/Polη translesion synthesis activity can be assayed using the published methods of Johnson, Prakash, and Prakash (1999) Science 283:1001-1004.

Briefly, cell extracts or purified RAD30/Polη from putative transgenic events are used in DNA polymerase activity assays using damaged DNA as a template. DNA synthesis is compared between DNA templates of identical sequence, wherein the control template comprises undamaged DNA and the experimental template comprises a Tˆ T dimer. A second, shorter ³²P-labeled oligonucleotide is annealed to the template in order to prime the DNA synthesis reaction. The products of the reaction are separated by SDS-Urea polyacrylamide gel electrophoresis (PAGE) and visualized by exposure to x-ray film. A phosphoimager with appropriate software can be used to quantify the polymerase activity. Percent activity can be determined from the number of nucleotides incorporated during the DNA synthesis reaction. The products synthesized from an unlabelled primer can be subjected to DNA sequence analysis using standard protocols to verify the accuracy of the DNA synthesized.

This type of assay can be run in either a “standing start” or “running start” format. In the standing start format, the primer is annealed to the template so that the 3′ hydroxyl group of the primer is located just before the Tˆ T dimer. In a running start assay, the 3′ hydroxyl of the primer is located 15 nucleotides upstream of the Tˆ T dimer.

The assay can be conducted in 10 μl reactions containing 25 mM KPO₄, pH 7.0; 5 mM MgCl₂; 5 mM dithiothreitol; bovine serum albumin (100 μg/ml); 10% glycerol; 100 μM dNTP; 10 nM of 5′ P³²-labelled primer annealed to template; and 2.5 nM RAD30/Polη. The reactions are incubated 5 min at 30° C., the reaction is terminated by the addition of 50 mM EDTA,1% SDS, and proteinase K (200 ng/ml) and placed at 55° C. for 30 min. The DNA can be precipitated by the addition of 10 μg herring sperm DNA; 300 mM sodium acetate; and 3 volumes of 95% ethanol. After the supernatant is removed, the precipitate is dried under vacuum, then resuspended in sample buffer for SDS-Urea PAGE.

### EXAMPLE 9

This example describes several illustrations of targeted genetic modification using RAD30/Polη. The Tˆ T translesion synthesis activity of RAD30/Polη, coupled with a modification template comprising at least one Tˆ T dimer, can be used to introduce targeted modifications to any polynucleotide sequence of interest. The modification template can be comprised of DNA, or can be a DNA-RNA chimera or other modified nucleotide polymer. These modifications can be used to enhance or suppress the expression of the DNA sequence of interest. The modifications can be the introduction of point or frameshift mutations in a sequence. Either one or two nucleotides can be inserted or converted by each Tˆ T dimer in the modification template. These targeted modifications can be used introduced in the UTR, regulatory sequences or the coding sequence. If introduced into the coding sequence, these modifications could be targeted to either exons or introns. Point mutations can be introduced to convert a codon to a more preferred codon, convert a codon to substitute a different amino acid, convert a codon to introduce a premature stop codon, alter an intron-exon splicing site or any other post-transcriptional processing site, or to alter other regulatory regions such as a promotor or any other UTR. Frameshift mutations can also be generated by the insertion of 1-2 adenines for every Tˆ T dimer in the modification template. More than one site in a target could be modified by designing modification templates comprising more than one Tˆ T dimer, or by using more than one template. The modification template can range anywhere between about 15 nucleotides in length to the full-length of the target polynucleotide of interest, typically the template will be between 15-200 nucleotides in length. The modification template is directed to the target polynucleotide by the shared homology between the sequences, typically the sequences will be identical except where a Tˆ T dimer is incorporated. Modifications to the target polynucleotide sequence could be used to enhance expression of target gene product, or can be used to suppress or knock-out expression of the target gene.

Any suitable method can be used to introduce the modification template to a cell comprising a target polynucleotide of interest, such as a particle-mediated method, or many other methods. The modification template can be delivered simultaneously with a RAD30/Polη polynucleotide or polypeptide, or can be delivered into a stably transformed cell comprising an introduced RAD30/Polη polynucleotide.

A. Introduction of a Point Mutation in a DNA Target

Any one or two adjacent nucleotides of a DNA target can be converted to adenine (A) by creating a modification template completely homologous to the target sequence except for the incorporation of a Tˆ T dimer positioned opposite the desired target site. Examples of this are illustrated below, with the target nucleotides shown in bold, and the Tˆ T dimer underlined. Dashes indicate other homologous nucleotides.

i. One Nucleotide Point Mutation:

Target: ---ATGCATGC---

Template: ---TACGTˆ TCG---

Product: ---ATGCAAGC---

ii. Two Nucleotide Point Mutation:

Target: ---ATGCATGC---

Template: ---TATˆ TTACG---

Product: ---ATAAATGC---

B. Introduction of a Frameshift Mutation in a DNA Target

One or two adjacent adenines can be inserted into the sequence of a DNA target by creating a modification template completely homologous to the target sequence except for the incorporation of a Tˆ T dimer inserted opposite the desired target site. Examples of this are illustrated below, with the target nucleotides shown in bold, and the Tˆ T dimer underlined. Insertion points are indicated by an asterick (*) Dashes indicate other homologous nucleotides.

i. One Nucleotide Insertion:

Target: ---ATGCA*TGC---

Template: ---TAGCTˆ TACG---

Product: ---ATGCAATGC---

ii. Two Nucleotide Insertion:

Target: ---AT**GCATG---

Template: ---TATˆ TCGTAC---

Product: ---ATAAGCATG---

### EXAMPLE 10

This example describes several illustrations of vector construction to produce polynucleotide constructs expressing RAD30/Polη polypeptides. Any standard molecular biology reference, such as Current Protocols in Molecular Biology, Vol. 1-3, Eds. Ausubel et al., a joint venture between Greene Publishing Associates, Inc. and John Wiley & Sons, Inc. (1994); Guide to Molecular Cloning Techniques, Methods in Enzymology, Vol. 152, Berger and Kimmel, Academic Press, Inc., San Diego, Calif. (1987); and Molecular Cloning—A Laboratory Manual, 2nd ed., Vol. 1-3, Sambrook et al., (1989) provides guidance on the molecular biological techniques and manipulations needed to construct vectors comprising the RAD30/Polη polynucleotide of SEQ ID NO: 1 or which express the RAD30/Polη polypeptide of SEQ ID NO: 2, or to produce fragments or variants of SEQ ID NOS: 1 or 2.

A. Vectors for Protein Expression

Proteins can be expressed in prokaryotic or eukaryotic expression systems. Further, vectors can be used which facilitate expression and or purification of the desired protein product by use of fusion partners such as GST or histidine tags.

i. Cloning Strategy for RAD30/Polη in pGEX6p GST E. coli Expression Vector

The unique restriction sites Cfr10I and NotI are present at the start of the coding sequence and after the stop codon, respectively, in RAD30/Polη. To facilitate cloning into a pGEX6p GST vector (Amersham Biosciences, Piscataway, N.J.), PCR primers can be designed to modify the expression vector to make it compatible with the available sites flanking the coding region in SEQ ID NO: 1. A PCR primer set with homology to the GST sequence (forward primer, GSTFOR 5′ TCCAAAAGAGCGTGCAGAGA 3′ shown in SEQ ID NO: 4) and to the PreScission protease (Amersham, Piscataway, N.J.) sequence (reverse primer, pGEXCFR 5′ GCTGGGACCGGCATGGGCCCCTGGAACAGAAC 3′ shown in SEQ ID NO: 5) can be used. The Cfr10I restriction site is added at the 3′ end of the reverse primer in order to introduce this cloning site. PCR amplification of the pGEX6p vector with this primer pair is used to introduce the Cfr10I cloning site. After amplification, the PCR product of about 500 bp is digested with Sful and Cfr10I and separated on an agarose gel and purified from the gel with the Qiagen gel extraction kit (Qiagen) to produce the RAD30/Polη linker of about 300 bp. The vector backbone is produced by a restriction enzyme digest of pGEX6p with the enzymes SfuI and NotI, and separated on an agarose gel and the about 4600 bp band purified with the Qiagen gel extraction kit. Finally, the RAD30/Polη coding region vector component is produced by digesting the RAD30/,Polη containing clone CMTMX27 (SEQ ID NO: 1) with Cfr10I and NotI restriction enzymes to produce the insert. The digest was gel purified using the Qiagen kit as above to yield the ˜2000 bp RAD30/Polη insert. The three purified components, the linker, the pGEX6p backbone, and the RAD30/Polη insert, are mixed together and ligated using a standard protocol to produce the RAD30/Polη pGEX6p GST E. coli expression vector, which is transformed into E. coli. Colonies on agar plates that showing amp resistance are selected and grown overnight in liquid media. RAD30/Polη plasmid is purified from these cultures using a standard miniprep kit or protocol. The RAD30/Polη construction is verified by restriction enzyme digestion with PstI and Sa/I enzymes. The digest is run on an agarose gel to visualize the products. Clones with the expected bands at 4000, 1300, 1000, and 650 bp are designated as RAD30/Polη pGEX6p GST clones. One of these clones can be further verified by sequence analysis using the sequencing primer pGEX5 (5′ GGGCTGGCAAGCCACGTTTGGTG 3′ shown in SEQ ID NO: 6). Sequence analysis confirms that RAD30/Polη is fused in frame with GST. Cultures of E. coli transformed with this plasmid and induced with IPTG express a protein of ˜97 kDa as expected from the RAD30/Polη-GST fusion product.

ii. Cloning Strategy for RAD30/Polη in pICZ GST Pichia Expression Vector

The RAD30/Polη pGEX6p GST E. coli expression vector from section (i) above is used to generate a Pichia pastoris GST fusion expression vector. The pICZ-GST vector (InVitrogen, Carlsbad, Calif.) is digested with Asp700 and NotI restriction enzymes to generate the pICZ-GST backbone of ˜3700 bp which is gel purified as above. The RAD30/Polη pGEX6p GST vector is first digested with NotI, and then that product is partially digested with Asp700 to generate the RAD30/Polη insert of ˜2400 bp which is gel purified. The purified digestion products are mixed, ligated for 2 hours to produce the RAD30/Polη pICZ GST, and then transformed into E. coli. Transformed colonies are selected for zeocin resistance. Select zeocin resistant colonies are grown overnight in liquid culture and purified plasmid preparations subjected to restriction enzyme digestion. Putative RAD30/Polη pICZ GST clones can be further confirmed by sequence analysis using the pGEX5 primer (SEQ ID NO: 6) as described above to confirm the Pichia pastoris RAD30/Polη GST expression vector.

iii. Cloning Strategy for RAD30/Polη in pMBAD His-6 E. coli Expression Vector

For this cloning of RAD30/Polh, a parent vector needs to be constructed. The parent vector is constructed by modifying the pBAD-A His-6 vector (InVitrogen, Carlsbad, Calif.) to facilitate the insertion of the RAD30/Polη coding region. The pBAD-A His-6 vector is modified by removing the multiple cloning site, and creating a replacement linker molecule inserted back into that site to create pMBAD. The restriction enzyme sites NcoI and NotI are unique sites, and can be used in the linker to accept the RAD30/Polη insert. The following primer pair, containing these sites, is used to amplify the RAD30/Polη coding sequence:

PNF or: 5′ GTACGTGCCATGGGGATGCCGGTTGCTAGGCCG 3′ (SEQ ID NO: 7) PNRev 5′ CGCCGATGCGGCCGCCTAAGACCTCGCGGGTGG 3′ (SEQ ID NO: 8)

Following the PCR reaction, the products are separated by agarose gel electrophoresis and the expected band of about 1900 bp is excised and purified suing the Qiagen gel extraction kit. The PCR product and the pMBAD vector are digested with NcoI and NotI to produce the RAD30/Polη ˜1900 bp insert, and the PMBAD ˜4000 bp vector backbone. These fragments are gel purified, mixed, ligated together and used to transform E. coli. Plasmid preps are done on select transformed colonies, and the purified plasmids digested with NcoI and NotI to confirm the presence of the 1900 bp RAD30/Polη insert. A plasmid preparation containing the proper insert is transformed into LMG194 cells (InVitrogen, Carlsbad, Calif.) and protein expression induced by arabinose. A protein of the expected size, 72 kDa, was observed on stained polyacrylamide gels.

iv. Cloning Strategy for RAD30/Poln Vectors for Yeast Complementation Tests

Four vectors from the pRS416 series of E. coli yeast shuttle vectors are available (pRS416 is deposited as GenBank Accession U03450). These vectors differ only by the promoter used to modulate protein expression. The four promoters used and some features are as follows:

Met25 promoter—repressed by methionine

Gall promoter—strongly repressed by glucose, highly induced by galactose

GalS promoter—strongly repressed by glucose, moderately induced by galactose

GalL promoter—strongly repressed by glucose, moderately induced by galactose

To introduce the RAD30/Polη coding region, the pRS416 vectors need compatible restriction sites. This can be achieved by digesting the pRS416 vectors with XhoI and XbaI, then creating a linker containing the desired restriction sites, such as EcoRI and NotI, and having overhanging ends compatible with XhoI and XbaI, and ligating this linker into the digestion product above. The linker molecular is created using two synthetic complementary single stranded primers (Sigma Chemical Co., St. Louis, Mo.) shown below:

YLTOP (SEQ ID NO: 9)

5′ TCGAGGCGGTGGCGGCCGCTCGTGGATCCCGTCGACCAGGAATTCGT 3′

YLBOTTOM (SEQ ID NO: 10) 5′CTAGACGAATTCCTGGTCGACGGGATCCACGAGCGGCCGCCACCGCC 3′

The primers are annealed by heating to 95° C. for 10 min., then slow cooling over a period of one hour. Once annealed, the primers have overhangs on each end that base pair with the XhoI and XbaI sites in the digested vector.

To minimize time, this linker can be inserted in one pRS416 vector, such as Met25pRS416 first. RAD30/Polη insert is prepared by digesting with NotI, followed by a partial digest with EcoRI. The expected 2000 bp band is gel purified using the gel extraction kit from Qiagen. A similar digestion is carried out on the Met25pRS416 vector, and the expect band at ˜4800 bp gel purified. The purified components are ligated together and transformed into E. coli. Plasmid preps from transformed colonies are digested with XhoI and XbaI to produce the RAD30/Polη insert of about 2100 bp, which is gel purified and then cloned into the other three pRS416 complementation vector backbones produced by a comparable digest.

### EXAMPLE 11

This is an example of a yeast complementation test. Any standard molecular biology reference, such as Current Protocols in Molecular Biology, Vol.1-3, Eds. Ausubel et al., a joint venture between Greene Publishing Associates, Inc. and John Wiley & Sons, Inc. (1994) can be used for guidance regarding yeast complementation testing.

The RAD30/Polη pRS416 vectors from Example 10 can be used to test for complementation of yeast S. cerevisiae RAD30 knockout strains. Four RAD30 yeast knockout strains are available from the American Type Culture Collection (ATCC), these strains are open reading frame deletions (ORF) of YDR419W on chromosome 4 of S. cerevisiae. A mating type a (ATCC 4004255), a mating type alpha (ATCC 4014255), a heterozygous diploid (ATCC 4024255), and a homozygous diploid (ATCC 4034255) strain are each available.

The four yeast complementation vectors from Example 10, along with an empty Met25pRS416 control vector are transformed into S. cerevisiae RAD30 knockout strain ATCC 4004255 using the yeast transformation protocol of Schiestl et al. (1993). To test the ability of RAD30/Polh (SEQ ID NO: 1) to complement the function of the S. cerevisiae gene, an UV radiation survival curve can be produced. This is done by growing the transformed yeast overnight and then plating a known number of cells on galactose containing media. The plated cells are exposed to a known level of UV radiation, keeping a duplicate plate from each vector type as a non-irradiated control. Wild-type S. cerevisiae (YDR419, ATCC) is used as a control (Schiestl et al., 1993). The plates are scored by counting the number of colonies on each UV irradiated plant and determining the percent that survive compared to the non-irradiated control. Similar experiments are done using plasmids with different yeast promoters, and the appropriate control vectors.

The above examples are provided to illustrate the invention but not to limit its scope. Other variants of the invention will be readily apparent to one of ordinary skill in the art and are encompassed by the appended claims. All publications, patents, patent applications, and computer programs cited herein are hereby incorporated by reference.

What is claimed is:

1. An isolated polynucleotide comprising a member selected from the group consisting of:
(a) a RAD30/Polη polynucleotide having at least 80% sequence identity to the polynucleotide of SEQ ID NO: 1, wherein the % sequence identity is based on the entire region coding for SEQ ID NO: 2 and is calculated by the GAP algorithm under default parameters;
(b) a RAD30/Polη polynucleotide encoding the polypeptide of SEQ ID NO: 2;
(c) a RAD30/Polη polynucleotide amplified from a Zea mays nucleic acid library using primers which selectively hybridize, under stringent hybridization conditions, to the polynucleotide of SEQ ID NO: 1;
(d) a RAD30/Polη polynucleotide which selectively hybridizes, under stringent hybridization conditions and a wash in 0.1×SSC at 60° C., to the complement of SEQ ID NO: 1, wherein stringent hybridization conditions comprise 50% formamide, 1 M NaCl, and 1% SDS at 37° C., or conditions equivalent thereto;
(e) the RAD30/Polη polynucleotide of SEQ ID NO: 1; and
(f) a polynucleotide which is fully complementary to a RAD30/Polη polynucleotide of (a), (b), (c), (d), or (e);
wherein the polynucleotide of (a), (b), (c), (d), and (e) encode a polypeptide with translesion DNA synthesis activity.

2. A recombinant expression cassette, comprising a member of claim 1 operably linked to a promoter.

3. A non-human host cell comprising the recombinant expression cassette of claim 2.

4. A transgenic plant comprising an isolated polynucleotide of claim 1.

5. A transgenic plant of claim 4, wherein said plant is a monocot.

6. The transgenic plant of claim 4, wherein said plant is a dicot.

7. The transgenic plant of claim 4, wherein said plant is selected from the group consisting of: maize, soybean, safflower, sunflower, sorghum, canola, wheat, alfalfa, cotton, rice, barley, and millet.

8. A transgenic seed from the transgenic plant of claim 4.

9. The polynucleotide of claim 1, wherein the polynucleotide has at least 85% sequence identity to the polynucleotide of SEQ ID NO: 1.

10. The polynucleotide of claim 1, wherein the polynucleotide has at least 90% sequence identity to the polynucleotide of SEQ ID NO: 1.

11. The polynucleotide of claim 1, wherein the polynucleotide has at least 95% sequence identity to the polynucleotide of SEQ ID NO: 1.

12. A method of modulating the level of RAD30/Polη in a plant cell, comprising:
(a) introducing into a plant cell a recombinant expression cassette comprising a RAD30/Polη polynucleotide of claim 1 operably linked to a promoter;
(b) culturing the plant cell under plant cell growing conditions; and
(c) inducing expression of said polynucleotide for a time sufficient to modulate the level of RAD30/Polη in said plant cell.

13. A method of modulating the level of RAD30/Polη in a plant, comprising:
(a) introducing into a plant cell a recombinant expression cassette comprising a RAD30/Polη polynucleotide of claim 1 operably linked to a promoter;
(b) culturing the plant cell under plant cell growing conditions;
(c) regenerating a plant which possesses the transformed genotype; and
(d) inducing expression of said polynucleotide for a time sufficient to modulate the level of RAD30/Polη in said plant.

14. The method of claim 13, wherein the level of Rad30/Polη is decreased in the plant.

15. The method of claim 13, wherein the level of Rad30/Polη is increased in the plant.

16. The method of claim 13, wherein the plant is maize, soybean, safflower, sunflower, sorghum, canola, wheat, alfalfa, cotton, rice, barley, and millet.

17. An isolated RAD30/Polη protein comprising a member selected from the group consisting of:
(a) a polypeptide of at least 30 contiguous amino acids from the polypeptide of SEQ ID NO: 2;
(b) the polypeptide of SEQ ID NO: 2;
(c) a polypeptide having at least 70% sequence identity to, and having at least one linear epitope in common with, the polypeptide of SEQ ID NO: 2, wherein said sequence identity is determined over the entire length of SEQ ID NO: 2 using the GAP program under default parameters; and
(d) at least one polypeptide encoded by a member of claim 1.

18. A method of introducing a heritable targeted polynucleotide sequence modification in a plant cell comprising:
(a) introducing into a plant cell a modification template for a target polynucleotide of interest and RAD30/Polη, wherein the modification template comprises a polynucleotide comprising at least one Tˆ T dimer at a specific site within its sequence to introduce at least one modification into the target polynucleotide of interest and wherein the modification template is targeted to the target polynucleotide of interest by having shared homology between the template and target; and
(b) culturing the plant cell under conditions sufficient to introduce a heritable sequence modification in the target polynucleotide of interest.

19. The method of claim 18, wherein the RAD30/Polη introduced into the plant cell comprises a polynucleotide.

20. The method of claim 18, wherein the RAD30/Polη introduced into the plant cell comprises a polypeptide.

21. The method of claim 19, wherein RAD30/Polη is stably transformed into the genome of the plant cell.

22. The method of claim 18 wherein the plant cell is from a monocot or a dicot.

23. The method of claim 22 wherein the plant cell is selected from the group consisting of: maize, soybean, safflower, sunflower, sorghum, canola, wheat, alfalfa, cotton, rice, barley, and millet.

24. A transformed plant cell produced by the method of claim 18.

25. The plant cell of claim 24, wherein the plant cell is from a monocot or a dicot.

26. The plant cell of claim 25 wherein the plant cell is selected from the group consisting of: maize, soybean, safflower, sunflower, sorghum, canola, wheat, alfalfa, cotton, rice, barley, and millet.

27. The method of claim 18, wherein the transformed plant cell is grown under conditions sufficient to produce a transformed plant.

28. A transformed plant produced by the method of claim 27.

29. The plant of claim 28 wherein the plant is from a monocot or a dicot.

30. The plant of claim 29 wherein the plant is selected from the group consisting of: maize, soybean, sunflower, safflower, sorghum, canola, wheat, alfalfa, cotton, rice, barley, and millet.

31. A transgenic seed produced by the plant of claim 28.

32. The method of claim 18, wherein the RAD30/Polη and the modification template are introduced into the plant cell simultaneously.

33. The method of claim 18, wherein the RAD30/Polη is introduced into the plant cell prior to the introduction of the modification template.

34. A method of modulating DNA repair in a plant comprising:
(a) introducing into a plant cell a recombinant expression cassette comprising a RAD30/Polη polynucleotide of claim 1 operably linked to a promoter;
(b) culturing the plant cell under plant cell growing conditions;
(c) regenerating a plant which possesses the transformed genotype; and
(d) inducing expression of said polynucleotide for a time sufficient to modulate the level of DNA repair in said plant.

35. The method of claim 34, wherein DNA repair is increased in the plant.

36. The method of claim 34, wherein DNA repair is decreased in the plant.

37. The method of claim 34, wherein the plant cell is from a monocot or a dicot

38. The method of claim 37 wherein the plant cell is selected from the group consisting of: maize, soybean, safflower, sunflower, sorghum, canola, wheat, alfalfa, cotton, rice, barley, and millet.

39. A transformed plant cell produced by the method of claim 34.

40. The plant cell of claim 39, wherein the plant cell is from a monocot or a dicot.

41. The plant cell of claim 40, wherein the plant cell is selected from the group consisting of: maize, soybean, safflower, sunflower, sorghum, canola, wheat, alfalfa, cotton, rice, barley, and millet.

42. A transformed plant produced by the method of claim 34.

43. The plant of claim 42 wherein the plant is from a monocot or a dicot.

44. The plant of claim 43 wherein the plant is selected from the group consisting of: maize, soybean, sunflower, safflower, sorghum, canola, wheat, alfalfa, cotton, rice, barley, and millet.

45. A transgenic seed produced by the plant of claim 42.

46. An isolated nucleic acid comprising a Rad30/Polη polynucleotide which encodes a polypeptide having at least 90% sequence identity over the entire length of SEQ ID NO: 2 as determined by the GAP algorithm under default parameters, wherein the polynucleotide encodes a polypeptide with translesion DNA synthesis activity.

47. The nucleic acid of claim 46, wherein the polynucleotide encodes a polypeptide having at least 95% sequence to SEQ ID NO: 2.

48. An isolated nucleic acid comprising a Rad30/Polη polynucleotide comprising at least 50 contiguous nucleotides of SEQ ID NO: 1, wherein the polynucleotide encodes a polypeptide having translesion DNA synthesis activity.

49. An isolated nucleic acid comprising a Rad30/Polη polynucleotide which encodes a polypeptide comprising at least 30 contiguous amino acids of SEQ ID NO: 2, wherein the polypeptide has translesion DNA synthesis activity.
